SEQUENCE LISTING (32 sequences)

SEQ ID NO: 1 - DNA, 1989 bp, canine distemper virus
atgcacaaggaaatccccgaaaaatccagaacccgaacacacacccaacaagacctcccccaacaaaaaagcaccgaatacaccgagatcaagacctcccgagcacgacacggcataacgccagctcagcgatccacgcactatggtcctcgaacattggacaggctcgtctgctacattatgaacagagccatgtcttgcaagcaagccagctacagatcggataacatcccagctcacggagaccacgagggtgttgtccatcacacaccagagagtgtctcccaaggagcgagatcccagctcaagaggcggacttccaatgcaatcaactcaggatttcagtacatctggttagtcctgtggtgcatcggaatagccagtctctttctttgttctaaggctcagatacattggaataatttatcaactattgggatcatcgggactgacagtgtccattataagatcatgactaggcccagccaccagtacttggtcataaaactaatgcctaatgtttcactcatagataattgtaccaaagcagaattaggtgagtatgagaaattattaaattcagtcctcgaaccaatcaaccaagctttgactctaatgaccaagaatgtgaagcctctacagtcagtagggtcaggtaggagacagaggcgttttgcaggagtggtgcttgcaggtgcagctttaggagtggccactgctgcacaaatcactgcagggatagctttacatcaatccaacctcaatgctcaagcaatccaatctctgagaactagccttgaacagtccaacaaggctatagaagaaattagggaggcaacccaggaaaccgtcattgccgttcagggagtccaggattacgtcaataatgaactcgtccctgctatacaacatatgtcgtgtgaattagttgggcagagattagggttaaaattgcttaggtattataccgagttgttgtcaatatttggcccgagtttacgtgatcctatttcagccgagatatcaattcaagcactgagttatgctcttgggggagaaattcataagatacttgagacgttggggtattctggaaatgatatgattgcaattttggagagtcgggggataaaaacaagaataactcatgttgatctccccgggaaatttatcatcttaagtatctcatacccaactttatcagaagtcaagggggttatagtccacagactggaagcagtttcttataacatagggtcacaagagtggtacaccactgtcccgaggtatgttgcaactaatggttacttaatatccaatttcgatgagtcatcctgtgtattcgtctcagaatcagccatttgtagccagaactccctataccccatgagcccacttctacaacaatgcattaggggcgacacttcatcttgtgctcggaccttggtgtctggaaccatgggcaataaatttattctgtcaaaaggcaatatcatcgcaaattgtgcttctatactatgtaagtgttatagcacaagcacaattatcaatcagagtcctgataagttgctgacctttattgcctccgatacatgcccactagttgaaatagatggtgtaaccatccaggttggaggcaggcaataccctgatatggtatacgaaagcaaagttgccctaggccctgctatatcacttgagaggctagatgtaggtacaaatttagggaacgcccttaagaaactggatgatgctaaggtactgatagactcatctaatcagatccttgagacggttaggcgctcttcctttaattttggcagtctcctcagcgttcctatattaagctgtgcagccctggctttgttattgctgatttactgctgtaaaagacgctatcaacagacactcaagcggaatactaaggtcgatccgacatttaaacctgatctgactgggacttctaaatcctatgtaagatcactctga

SEQ ID NO: 2 - protein, 662 aa, canine distemper virus
MHKEIPEKSRTRTHTQQDLPQQKSTEYTEIKTSRARHGITPAQRSTHYGPRTLDRLVCYIMNRAMSCKQASYRSDNIPAHGDHEGVVHHTPESVSQGARSQLKRRTSNAINSGFQYIWLVLWCIGIASLFLCSKAQIHWNNLSTIGIIGTDSVHYKIMTRPSHQYLVIKLMPNVSLIDNCTKAELGEYEKLLNSVLEPINQALTLMTKNVKPLQSVGSGRRQRRFAGVVLAGAALGVATAAQITAGIALHQSNLNAQAIQSLRTSLEQSNKAIEEIREATQETVIAVQGVQDYVNNELVPAIQHMSCELVGQRLGLKLLRYYTELLSIFGPSLRDPISAEISIQALSYALGGEIHKILETLGYSGNDMIAILESRGIKTRITHVDLPGKFIILSISYPTLSEVKGVIVHRLEAVSYNIGSQEWYTTVPRYVATNGYLISNFDESSCVFVSESAICSQNSLYPMSPLLQQCIRGDTSSCARTLVSGTMGNKFILSKGNIIANCASILCKCYSTSTIINQSPDKLLTFIASDTCPLVEIDGVTIQVGGRQYPDMVYESKVALGPAISLERLDVGTNLGNALKKLDDAKVLIDSSNQILETVRRSSFNFGSLLSVPILSCAALALLLLIYCCKRRYQQTLKRNTKVDPTFKPDLTGTSKSYVRSL

SEQ ID NO: 3 - protein, 75 aa, canine distemper virus
MNRAMSCKQASYRSDNIPAHGDHEGVVHHTPESVSQGARSQLKRRTSNAINSGFQYIWLVLWCIGIASLFLCSKA

SEQ ID NO: 4 - protein, 31 aa, artificial
  synthetic polypeptide
MTSNAINSGFQYIWLVLWCIGIASLFLCSKA

SEQ ID NO: 5 - protein, 26 aa, artificial
  synthetic polypeptide
MSIMGLKVNVSAIFMAVLLTLQTPTG

SEQ ID NO: 6 - protein, 28 aa, artificial
  synthetic polypeptide
MAINSGSQCTWLVLWCLGIASLFLCSKA

SEQ ID NO: 7 - protein, 28 aa, artificial
  synthetic polypeptide
MAINSGSQCTWLVLWCLGMASLFLCSKA

SEQ ID NO: 8 - protein, 28 aa, artificial
  synthetic polypeptide
MAINSGSQCTWLVLWCFGTASLFLCSKA

SEQ ID NO: 9 - protein, 28 aa, artificial
  synthetic polypeptide
MATSPGPQCTWLVLWCIGIASLFLCSEA

SEQ ID NO: 10 - protein, 28 aa, artificial
  synthetic polypeptide
MAVKSGSQCTWLVLWCIGVASLFLCSKA

SEQ ID NO: 11 - protein, 28 aa, artificial
  synthetic polypeptide
MASNPGSQCTWLVLWCIGIASLFLCSKA

SEQ ID NO: 12 - protein, 28 aa, artificial
  synthetic polypeptide
MATNAGSQYTWLVLWCIGIASLFLCSKA

SEQ ID NO: 13 - protein, 28 aa, artificial
  synthetic polypeptide
MATSSGSQCTWLVLWCIGVASLFLCSKA

SEQ ID NO: 14 - protein, 28 aa, artificial
  synthetic polypeptide
MAINSGSQCTWLVLWCIGVASLFLCSKA

SEQ ID NO: 15 - protein, 28 aa, artificial
  synthetic polypeptide
MAINSGFQYIWLVLWCIGIASLFLCSKA

SEQ ID NO: 16 - DNA, 1824 bp, canine distemper virus
atgctctcctaccaagacaaggtgggtgccttctacaaggataatgcaagagctaatttgtccaagctgtccttagtgacagaagagcaagggggcaggagaccaccctatttgctgtttgtccttctcatcctactggttggaatcctggccttgcttgctatcactggagttcgatttcaccaggtatcaattagcaatatggaatttagcagattgttgaaagaggatatggagaagtcagatgccgtacatcaccaagtcatagatgtcttgacaccgctcttcaaaattattggagatgagattgggttacggttgccacaaaaactaaacgagatcaaacaatttatccttcaaaagactaacttcttcaatccgaacagggaattcgacttccgcgatctccactggtgcattaatccgcctagtaagatcaaagtgaattttactagttactgtgatacaattgggatcagaaaatccattgcattggcagcaaatcccatccttttttcagcactctccggaggcagaggtgacatattcccaccatacagatgcagtggagctactacttcagttggcaaatctttccccctatcagtatcattatccatgtctttgatctcaagaacatcagagataatcaatatgctgacctctatctcagacggagtgtatggtaaaacttatttgctagtgcctgattatattgaaggggagttcgacacgcaaaagattcgagtctttgagatagggttcatcaaaaggtggctgaatgacatgccattattccagacaaccaactatatgatcctcccggagaattctaaaaccaaagtatgtactatagcagtgggcgagttgacactggcttccttgtgtgtagatgagagcactgtattattatatcatgacagcaatgattcacaagatggtattctagtagtgacgctgggaatctttggggcaacacctatggatcaagtcgaagaggtgatacctgtcgctcacccatcagtagaaaaaatacatataacaaatcaccgtggtttcataaaagattcggtagcaacctggatggtgcctgcattggtctctgagaacctagaggaacaaaaaaattgtctggagtcggcttgtcaaagaaaatcctaccctatgtgcaaccaaacatcatgggaaccctttggaggaggacagttgccatcttatgggcggttgacgttacatctagatgcaagcactgaccgtcaacttaacatatcatttacgtacggtccggttatactgaatggaaatggtatggattattatgaaagcccacttttggactccggatggcttaccattcctcctaagaatggaacaattcttggattgataaacaaagcaagtagaggagaccagttcactgtgatccctcatgtgttgtcatttgcgcccagggaatcaagtggaaactgttatttacctattcaaacatcccagattatggataaagatgtccttactgagtctaatttagttgtgttgcctacacagaattttagatatgtcatagcaacatatgatatatcccgaaacgatcatgcgattgtttattatgtttatgacccaatccggacgatttcttatacgtacccatttagactaactaccaagggtagatctgatttcctaaggattgaatgttttgtgtgggatgacgacctgtggtgtcaccaattttacaggttcgaggctgacatcaccaactctacaaccagtgttgagaatttagtccgtataagattctcctgtaaccgttcaaaaccttga

SEQ ID NO: 17 - protein, 607 aa, canine distemper virus
MLSYQDKVGAFYKDNARANLSKLSLVTEEQGGRRPPYLLFVLLILLVGILALLAITGVRFHQVSISNMEFSRLLKEDMEKSDAVHHQVIDVLTPLFKIIGDEIGLRLPQKLNEIKQFILQKTNFFNPNREFDFRDLHWCINPPSKIKVNFTSYCDTIGIRKSIALAANPILFSALSGGRGDIFPPYRCSGATTSVGKSFPLSVSLSMSLISRTSEIINMLTSISDGVYGKTYLLVPDYIEGEFDTQKIRVFEIGFIKRWLNDMPLFQTTNYMILPENSKTKVCTIAVGELTLASLCVDESTVLLYHDSNDSQDGILVVTLGIFGATPMDQVEEVIPVAHPSVEKIHITNHRGFIKDSVATWMVPALVSENLEEQKNCLESACQRKSYPMCNQTSWEPFGGGQLPSYGRLTLHLDASTDRQLNISFTYGPVILNGNGMDYYESPLLDSGWLTIPPKNGTILGLINKASRGDQFTVIPHVLSFAPRESSGNCYLPIQTSQIMDKDVLTESNLVVLPTQNFRYVIATYDISRNDHAIVYYVYDPIRTISYTYPFRLTTKGRSDFLRIECFVWDDDLWCHQFYRFEADITNSTTSVENLVRIRFSCNRSKP

SEQ ID NO: 18 - protein, 34 aa, measles virus
MSPQRDRINAFYKDNPHPKGSRIVINREHLMIDR

SEQ ID NO: 19 - protein, 16 aa, artificial
  synthetic polypeptide
MGSRIVINREHLMIDR

SEQ ID NO: 20 - protein, 10 aa, artificial
  synthetic polypeptide
MNREHLMIDR

SEQ ID NO: 21 - protein, 34 aa, canine distemper virus
MLSYQDKVGAFYKDNARANLSKLSLVTEEQGGRR

SEQ ID NO: 22 - protein, 14 aa, artificial
  synthetic polypeptide
MKLSLVTEEQGGRR

SEQ ID NO: 23 - protein, 4 aa, artificial
  synthetic polypeptide
MGRR

SEQ ID NO: 24 - protein, 135 aa, canine distemper virus
MHKEIPEKSRTRTHTQQDLPQQKSTEYTEIKTSRARHGITPAQRSTHYGPRTLDRLVCYIMNRAMSCKQASYRSDNIPAHGDHEGVVHHTPESVSQGARSQLKRRTSNAINSGFQYIWLVLWCIGIASLFLCSKA

SEQ ID NO: 25 - DNA, 25 bp, artificial
  synthetic oligonucleotide
agaaaacttagggctcaggtagtcc

SEQ ID NO: 26 - DNA, 23 bp, artificial
  synthetic oligonucleotide
tcgtctgtaagggatttctcacc

SEQ ID NO: 27 - DNA, 21 bp, artificial
  synthetic oligonucleotide
aggacatagcaagccaacagg

SEQ ID NO: 28 - DNA, 25 bp, artificial
  synthetic oligonucleotide
ggactacctgagccctaagttttct

SEQ ID NO: 29 - DNA, 64 bp, artificial
  synthetic oligonucleotide
ccggtagttaattaaaacttagggtgcaagatcatcgataatgctctcctaccaagataaggtg

SEQ ID NO: 30 - DNA, 90 bp, artificial
  synthetic oligonucleotide
ctatttcacactagtgggtatgcctgatgtctgggtgacatcatgtgattggttcactagcagcctcaaggttttgaacggttacaggag

SEQ ID NO: 31 - DNA, 168 bp, artificial
  synthetic oligonucleotide
cccggtagttaattaaaacttagggtgcaagatcatcgataatgtcaccacaacgagaccggataaatgccttctacaaagataacccccatcccaagggaagtaggatagtcattaacagagaacatcttatgattgatagaccaccctatttgctgtttgtccttc

SEQ ID NO: 32 - DNA, 76 bp, artificial
  synthetic oligonucleotide
gcgaagactgacggtccccccaggagttcaggtgctgggcacggtgggcaaggttttgaacggttacaggagaatc

(19) United States
(12) Patent Application Publication
(10) Pub. No.: US 2024/0141376 A1
(43) Pub. Date: May 2, 2024

(54) ENGINEERING HEMAGGLUTININ AND FUSION POLYPEPTIDES OF CANINE DISTEMPER VIRUS

(71) Applicant: Mayo Foundation for Medical Education and Research, Rochester, MN (US)

(72) Inventors: Stephen J. Russell, Rochester, MN (US); Miguel A. Munoz Alia, Rochester, MN (US)

(21) Appl. No.: 18/278,564

(22) PCT Filed: Feb. 23, 2022
(86) PCT No.: PCT/US2022/017436

§ 371 Date: Aug. 23, 2023

Related U.S. Application Data
(60) Provisional application No. 63/152,682, filed on Feb. 23, 2021.

(51) Int. Cl. C12N 15/86; C07K 14/005

(52) U.S. Cl. CPC C12N 15/86; C07K 14/005; C12N 2740/15045; C12N 2740/16045; C12N 2760/18422

(57) ABSTRACT

Canine distemper vims (CDV) hemagglutinin (H) and fusion (F) polypeptides are provided herein. For example, engineered configurations of CDV fusogenic membrane glycoprotein (FMG) complexes containing H and F glycoproteins are provided herein, as are pseudotyped viruses (e.g., pseudotyped lentiviruses) containing the engineered CDV FMG complexes on their surface. In addition, this document provides nucleic acid molecules encoding CDV-H and/or CDV-F polypeptide components, methods for making recombinant cells expressing the CDV-H and CDV-F polypeptides, and methods for making and using pseudotyped viruses (e.g., pseudotyped lentiviruses) containing CDV FMG complexes.

### CROSS-REFERENCE TO RELATED APPLICATIONS

This application claims priority from U.S. Provisional Application Ser. No. 63/152,682, filed Feb. 23, 2021.

### TECHNICAL FIELD

This document relates to canine distemper virus (CDV) hemagglutinin (H) and fusion (F) polypeptides. For example, this document relates to engineered configurations of CDV fusogenic membrane glycoprotein (FMG) complexes containing H and F glycoproteins, as well as pseudotyped viruses (e.g., pseudotyped lentiviruses) having engineered CDV FMG complexes on their surface. In addition, this document relates to nucleic acid molecules encoding CDV-H and/or CDV-F polypeptide components of a FMG complex, methods for making recombinant cells expressing CDV-H and CDV-F polypeptides, and methods for making and using pseudotyped viruses (e.g., pseudotyped lentiviruses) containing CDV FMG complexes to treat cancer or infectious diseases.

### BACKGROUND

Viruses such as vesicular stomatitis viruses (VSVs), measles viruses (MeVs), adenoviruses, and lentiviruses (LVs) can be used as oncolytic viruses to treat cancer. In addition, LVs have emerged as a key technology to deliver cell-type-specific medicinal products. LV contains a diploid, single-stranded, positive-sense RNA-genome that present in a complex with a nucleocapsid protein, and also is bound to reverse transcriptase, protease, and integrase polypeptides. The LV genome is organized from the 5′ to the 3′ end, and contains gag, pol, and env genes that encode major polypeptide components of the virus. In particular, gag encodes structural proteins, pol encodes the reverse transcriptase, protease and integrase, and env encodes the virus envelope glycoprotein.

MeV (also referred to as MV) is a single-stranded, negative-sense, enveloped, non-segmented RNA virus of the genus Morbillivirus within the family Paramyxoviridae. The MeV genome encodes six main polypeptides: a nucleoprotein (N) polypeptide, a phosphoprotein (P) polypeptide, a matrix (M) polypeptide, a fusion (F) polypeptide, a hemagglutinin (H) polypeptide, and an RNA dependent RNA polymerase (L) polypeptide, as well as the C and V non-structural proteins that serve as innate immunity antagonists. MV has a lipid membrane envelope, with which virion surface glycoproteins H and F are associated. The MeV F/H complex has been extensively engineered to retarget both virus entry and intercellular fusion. However, high prevalence of MV seropositivity in the human population has limited the application of this technology, whether for targeted fusogenic cancer therapy, targeted virotherapy, or targeted in vivo gene delivery.

Like MeV, CDV is classified in the genus Morbillivirus within the family Paramyxoviridae. CDV also has an unsegmented, single-stranded, negative-sense, RNA genome and an enveloped virus particle. The CDV genome encodes six main polypeptides: a matrix (M) polypeptide, a fusion (F) polypeptide, a hemagglutinin (H) polypeptide, a nucleocapsid (N) polypeptide, a polymerase (L) polypeptide, and a phosphoprotein (P) polypeptide. In contrast to MeV, human seropositivity to CDV is low. In addition, although MeV and CDV are closely related, targeting technologies successfully developed for the MeV F/H complex have not been readily transferrable to the CDV F/H complex.

### SUMMARY

This document provides methods and materials related to CDV-H and/or CDV-F polypeptides. For example, this document provides engineered configurations of CDV FMG complexes containing H and F glycoproteins, as well as pseudotyped viruses (e.g., lentiviruses) having engineered CDV FMG complexes on their surface. In addition, this document provides nucleic acid molecules encoding CDV-H and CDV-F polypeptide components of a FMG complex, methods for making recombinant cells expressing CDV-H and/or CDV-F polypeptides, and methods for making and using pseudotyped viruses (e.g., lentiviruses) containing CDV FMG complexes to treat cancer or infectious diseases.

As described herein, CDV-F polypeptides can be designed to have increased fusogenic activity when expressed by cells in combination with a CDV-H polypeptide as compared to the level of fusogenic activity of a wild-type CDV-F polypeptide expressed by comparable cells in combination with that CDV-H polypeptide. For example, CDV-F polypeptides designed to have a truncated signal peptide sequence can exhibit increased fusogenic activity when expressed by cells in combination with a CDV-H polypeptide (e.g., a wild-type or de-targeted CDV-H polypeptide) as compared to the level of fusogenic activity of a wild-type CDV-F polypeptide containing a full length signal peptide sequence expressed by comparable cells in combination with that CDV-H polypeptide. Such CDV-F polypeptides can be incorporated into a virus to create a recombinant virus having the ability to increase fusogenic activity observed in cells infected by that virus.

As also described herein, CDV-H polypeptides can be designed to be de-targeted such that they do not have the ability, when used in combination with an F polypeptide (e.g., a CDV-F polypeptide), to enter cells via, or fuse cells via, for example, a NECTIN4 polypeptide or, in some cases, a SLAMF1 polypeptide. In some cases, CDV-H polypeptides can be designed to be de-targeted such that they do have the ability, when used in combination with an F polypeptide (e.g., a CDV-F polypeptide), to enter cells via, or fuse cells via, a SLAMF1 polypeptide. CDV-H polypeptides described herein can provide a platform for designing H polypeptides having the ability to be re-targeted to one or more targets of interest. For example, an H polypeptide provided herein can be further engineered to contain a binding sequence (e.g., a single chain antibody (scFv) sequence) having binding specificity for a target of interest, such that a recombinant virus containing that re-targeted H polypeptide, and an F polypeptide, can infect cells expressing that target.

In addition, viruses such as LVs can be engineered to have CDV-H and/or F polypeptides on their surfaces, without carrying nucleic acids encoding the CDV-H and/or F polypeptides. Such pseudotyped LVs therefore contain a nucleic acid molecule containing the native gag, pol, and env genes, but can also have a CDV-F polypeptide (e.g., a wild-type CDV-F polypeptide or an engineered CDV-F polypeptide described herein) and a CDV-H polypeptide (e.g., a wild-type CDV-H polypeptide or an engineered CDV-H polypeptide described herein) on their envelope surface.

As described herein, pseudotyped LVs can be designed to have a preselected tropism. For example, CDV-F and/or -H polypeptides having knocked out specificity for NECTIN4 and/or SLAMF1 can be used. In such cases, a scFv or polypeptide ligand can be attached to, for example, the C-terminus of the CDV-H polypeptide. In such cases, the scFv or polypeptide ligand can determine the tropism of the pseudotyped LV. Examples of scFvs that can be used to direct pseudotyped LVs to cellular receptors (e.g., tumor associated cellular receptors) include, without limitation, anti-EGFR, anti-EGFRvIII, anti-alpha folate-receptor (αFR), anti-CD3, anti-CD46, anti-CD38, anti-HER2/neu, anti-EpCAM, anti-CEA, anti-CD20, anti-CD133, anti-CD117 (c-kit), anti-CD138, and anti-PSMA scFvs. Examples of polypeptide ligands that can be used to direct pseudotyped LVs include, without limitation, urokinase plasminogen activator uPA polypeptides, cytokines such as IL-13 or IL-6, single chain T cell receptors (scTCRs), echistatin polypeptides, stem cell factor (SCF), Flt3, EGF, and integrin binding polypeptides.

In some cases, a pseudotyped LV provided herein can have a nucleic acid molecule that includes a sequence encoding an interferon (IFN) polypeptide (e.g., a human IFN-β polypeptide), a sodium iodide symporter (NIS) polypeptide (e.g., a human NIS polypeptide), a fluorescent polypeptide (e.g., a GFP polypeptide), any appropriate therapeutic transgene (e.g., HSV-TK or cytosine deaminase), a polypeptide that antagonizes host immunity (e.g., influenza NS1, HSVγ34.5, or SOCS1), a toxin, a chimeric antigen receptor, or a tumor antigen (e.g., cancer vaccine components). The nucleic acid encoding the IFN polypeptide can be positioned in the nef-frame or in place of the env open reading frame in the LV genome. Such a position can allow the viruses to express an amount of the IFN polypeptide that is effective to activate anti-viral innate immune responses in non-cancerous tissues, and thus alleviate potential viral toxicity, without impeding efficient viral replication in cancer cells. The nucleic acid encoding the NIS polypeptide can be positioned in place of the env open reading frame. Such a position of can allow the viruses to express an amount of the NIS polypeptide that (a) is effective to allow selective accumulation of iodide in infected cells, thereby allowing both imaging of viral distribution using radioisotopes and radiotherapy targeted to infected cancer cells, and (b) is not so high as to be toxic to infected cells. Positioning the nucleic acid encoding an IFN polypeptide in the nef-frame and positioning the nucleic acid encoding a NIS polypeptide in place of the env open reading frame within the genome of a LV can result in LVs that are viable, that have the ability to replicate and spread, that express appropriate levels of functional IFN polypeptides, and that express appropriate levels of functional NIS polypeptides to take up radio-iodine for both imaging and radio-virotherapy.

In general, one aspect of this document features a pseudotyped virus. The pseudotyped virus can comprise, consist essentially of, or consist of a canine distemper virus (CDV) hemagglutinin (H) polypeptide and a CDV fusion (F) polypeptide, where the virus lacks nucleic acid encoding the H polypeptide and lacks nucleic acid encoding the F polypeptide. The virus can be a lentivirus (LV). The CDV-H polypeptide can include an amino acid substitution at one or more of positions 194, 195, 478, 479, 540, 544, and 548 according the amino acid numbering of SEQ ID NO:17. The CDV-H polypeptide can include V478L, L479D, T544S, and T548D substitutions according to the amino acid numbering of SEQ ID NO:17. The CDV-H polypeptide can include a D540G substitution according to the amino acid numbering of SEQ ID NO:17. The CDV-H polypeptide can include S194I, V195R, V478L, L479D, D540G, T544S, and T548D substitutions according to the amino acid numbering of SEQ ID NO:17. The CDV-H polypeptide can include a truncated N-terminal cytoplasmic domain, as compared to the sequence set forth in SEQ ID NO:17. The truncated N-terminal cytoplasmic domain can have a deletion that is 27 to 32 amino acids in length. The truncated N-terminal cytoplasmic domain can have the sequence set forth in SEQ ID NO:23. The CDV-F polypeptide can include the amino acid sequence set forth in SEQ ID NO:2. The CDV-F polypeptide can include a signal peptide sequence that is less than 75 amino acid residues in length. In some cases, the signal peptide sequence can include no more than 75 amino acid residues of SEQ ID NO:24. The CDV-F polypeptide can include SEQ ID NO:2, with the proviso that the CDV-F polypeptide lacks at least amino acid residues 1 to 60 or lacks at least amino acid residues 1 to 105 of SEQ ID NO:2. The virus can be lentivirus, and nucleic acid within the lentivirus can be disarmed. The virus can be a lentivirus, and the lentivirus can include exogenous nucleic acid encoding one or more of an interferon (IFN) polypeptide, a sodium iodide symporter (NIS) polypeptide, a toxin polypeptide, or a chimeric antigen receptor (CAR) polypeptide. The CDV-H polypeptide can include an amino acid sequence of a single chain antibody. The single chain antibody can be a single chain antibody that specifically binds to a CD19, CD20, CD38, CD46, CD117, EGFR, αFR, HER2/neu, PSMA, or EpCAM polypeptide.

In another aspect, this document features a composition containing a pseudotyped virus described herein.

In another aspect, this document features a CDV-H polypeptide having an amino acid substitution at one or more of positions 194, 195, 478, 479, 540, 544, and 548 according to the amino acid numbering of SEQ ID NO:17. The CDV-H polypeptide can include V478L, L479D, T544S, and T548D substitutions according to the amino acid numbering of SEQ ID NO:17. The polypeptide can have a D540G substitution according to the amino acid numbering of SEQ ID NO:17. The polypeptide can include S194I, V195R, V478L, L479D, D540G, T544S, and T548D substitutions according to the amino acid numbering of SEQ ID NO:17. The polypeptide can include a truncated N-terminal cytoplasmic domain, as compared to the sequence set forth in SEQ ID NO:17. The truncated N-terminal cytoplasmic domain can have a deletion that is 27 to 32 amino acids in length. The truncated N-terminal cytoplasmic domain can have the sequence set forth in SEQ ID NO:23. The CDV-H polypeptide can include an amino acid sequence of a single chain antibody. The single chain antibody can be a single chain antibody that specifically binds to a CD19, CD20, CD38, CD46, CD117, EGFR, αFR, HER2/neu, PSMA, or EpCAM polypeptide.

In another aspect, this document features a nucleic acid molecule encoding a CDV-H polypeptide described herein.

In still another aspect, this document features a composition containing a nucleic acid molecule described herein. The composition can further contain a nucleic acid molecule encoding a CDV-F polypeptide. The CDV-F polypeptide can include the amino acid sequence set forth in SEQ ID NO:2. The CDV-F polypeptide can include a signal peptide sequence that is less than 75 amino acid residues in length. In some cases, the signal peptide sequence can include no more than 75 amino acid residues of SEQ ID NO:24. The CDV-F polypeptide can include SEQ ID NO:2, with the proviso that the CDV-F polypeptide lacks at least amino acid residues 1 to 60 or lacks at least amino acid residues 1 to 105 of SEQ ID NO:2.

In another aspect, this document features a method for treating cancer. The method can comprise, consist essentially of, or consist of administering a composition provided herein to a mammal that contains cancer cells, where the number of cancer cells within the mammal is reduced following the administering. The mammal can be a human. The cancer can be myeloma, melanoma, glioma, lymphoma, mesothelioma, lung cancer, brain cancer, stomach cancer, colon cancer, rectum cancer, kidney cancer, prostate cancer, ovary cancer, breast cancer, pancreas cancer, liver cancer, or head and neck cancer.

This document also features a method for inducing tumor regression in a mammal. The method can comprise, consist essentially of, or consist of administering a composition provided herein to a mammal having a tumor, where the size of the tumor is reduced following the administering. The mammal can be a human. The cancer can be myeloma, melanoma, glioma, lymphoma, mesothelioma, lung cancer, brain cancer, stomach cancer, colon cancer, rectum cancer, kidney cancer, prostate cancer, ovary cancer, breast cancer, pancreas cancer, liver cancer, or head and neck cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a nucleic acid sequence (SEQ ID NO:1) of a CDV F open reading frame encoding a CDV F polypeptide (SEQ ID NO:2). FIG. 1B is an amino acid sequence of a CDV F polypeptide (SEQ ID NO:2).

FIG. 2A is a nucleic acid sequence (SEQ ID NO:16) of a CDV H open reading frame encoding a CDV H polypeptide (SEQ ID NO:17). FIG. 2B is an amino acid sequence of a CDV H polypeptide (SEQ ID NO:17).

FIG. 3 is a schematic illustrating a method for targeting lentivirus particles (LVs) to specific cell types using canine distemper glycoproteins (e.g., CDV-H and F polypeptides). Receptor targeting of LVs involves the substitution of the native envelope glycoprotein (env) on the HIV-derived vector by the heterologous CDV-F/H glycoproteins. Fully retargeting capabilities are achieved by elimination of natural tropism on CDV-F/H and display of a targeting domain.

FIGS. 4A-4G show that truncation of the CDV-H and F glycoproteins is not required for LVs pseudotyping. FIG. 4A is a schematic of a morbillivirus hemagglutinin polypeptide displaying a 6×H tag at the C-terminus. Amino acid sequences for wild-type and truncated versions of the measles virus and CDV hemagglutinin are shown below the schematic (MSPQRDRINAFYKDNPHPKGSRIVINREHLMIDR, SEQ ID NO:18; MGSRIVINREHLMIDR, SEQ ID NO:19; MNREHLMIDR, SEQ ID NO:20; MLSYQDKVGAFYKDNARANLSKLSLVTEEQGGRR, SEQ ID NO:21; MKLSLVTEEQGGRR, SEQ ID NO:22; MGRR, SEQ ID NO:23). CDV-HcMeV-H represents a CDV-H chimeric polypeptide with the MeV-H cytoplasmic domain. FIG. 4B includes a series of images and flow cytometry plots showing that truncation of the MeV-F/H cytoplasmic tail is required for efficient LVs pseudotyping. Jurkat cells were transduced with LVs displaying the indicated envelope polypeptides. The percentage of GFP positive cells was measured by flow cytometry four days after transduction. FIG. 4C includes a series of graphs plotting surface and total expression of native and CDV-H variants as determined by flow cytometry on HEK-293T cells transiently transfected with the indicated expression plasmid (filled curves) and compared to mock transfected cells (open curves). Cells were stained with PE-coupled anti-HIS antibody on either permeabilized (total) or fixed cells (surface). One representative experiment out of two biological replicates is shown. FIG. 4D is a graph plotting mean fluorescence intensities for the positively stained cell population. Bars represent the mean, and error bars represent the standard deviation from two independent experiments. FIG. 4E is a graph plotting the screening titers of unconcentrated LVs produced using the indicated CDV-F/H combinations. Titers were determined on CHO (gray bars) and CHO cells expressing canine slamf1 (CHO-slamf1) (n=2; mean±SD). FIG. 4F is a graph plotting relative titers of unconcentrated LVs produced by varying the ratio of CDV-F/H-expression plasmids. Titers were normalized to that obtained on CHO-slamf1 using a 1:1 ratio. n=2; mean±SD. FIG. 4G is a graph plotting the titers of MeV-FΔ30/HΔ24 and CDV-F/H-displaying LVs. Titers were determined on unconcentrated stocks produced using a F/H ratio of 1:1. n=2; mean±SD.

FIGS. 5A-5E demonstrate retargeting of LV particles displaying CDV-F/H glycoproteins. FIG. 5A is a depiction of the human SLAMF1 footprint on the MeV-H (left) and on CDV-H (right), showing a surface representation of the MeV-H (PBD: ALW) and CDV-H (model) with the residues buried by the interaction with human SLAMF1 shown in darker shading. The structure of CDV-H in complex with SLAMF1 was modeled using Pyre2. Residues on the CDV-H polypeptide substituted to their MeV-H counterpart for retargeting purposes are indicated in white. FIG. 5B is a graph plotting levels of transduction of VSV-G-displaying LVs in stably-expressing CHO cell lines. Starting at a 1:2 dilution, five-fold dilutions of supernatant-containing LVs particles were used to transduce the CHO cell panel. Seventy-two hours later, luminescence was measured in cell lysates. FIG. 5C is a series of graphs plotting transduction of CDV-F/H LVs displaying parental (left) or amino acid substituted glycoproteins. The P493S/Y539A substitutions in CDV-H eliminated tropism for canine nectin4 (middle), while the V478L/L479D/T544S/T548D (LDSD) substitutions in CDV-H eliminated tropism for canine nectin4 and canine slamf1 (right). FIG. 5D is a graph plotting tropism of EpCAM retargeted CDV-F/H-LVs. Ac1 represents an EpCAM-specific DARPin targeting ligand. FIG. 5E is a graph plotting the degree of tropism for human SLAMF1, canine slamf1, human NECTIN4, and canine nectin4 by CDV glycoproteins. CDV-F/H-LVs displaying a parental or substituted CDV-H polypeptide were used to transduce the indicated cell lines. All data are represented as the mean and SD of two independent experiments with at least biological replicates. Neither the D540G substitution in CDV-H nor the combination of S194I/V195R/V478L/L479D/T544S/T548D in CDV-H conferred tropism for human SLAMF1, but when the two sets of mutations were combined in CDV-H, tropism for human SLAMF1 was observed.

FIGS. 6A and 6B show that fully retargeted CDV-H Ac1 HIS/F LVs outperformed MeV-FΔ30/HΔ24Ac1 HIS LVs. FIG. 6A is an image showing western blot analysis of LVs particles for incorporation of MeV-HΔ24 in combination with MeV-FΔ30, MeV-HaalsΔ24HIS in combination with MeV-FΔ30, CDV-HLDSDHIS in combination with CDV-F, or CDV-HLDSDAc1 HIS in combination with CDV-F. 0.5 μg of LVs particles/lane were used and incorporation was detected with anti-HIS antibody (GenScript, Cat #A01857-40, 0.2 ug/mL) or anti-p24 antibody (Sino biological, Cat #11695-RP02 1:1000). FIG. 6B is a graph plotting receptor-dependent transduction of pseudotyped LVs on the parental CHO cell line (K1) or CHO cells stably expressing anti-HIS, CD46, human SLAMF, canine SLAMF1, canine nectin4, EpCAM or CD38. Luminescence expression was determined 72 hours post transduction and values were normalized based on the levels obtained in CHO-αHIS. Data are represented as mean±SD of a representative experiment with two biological replicates.

### DETAILED DESCRIPTION

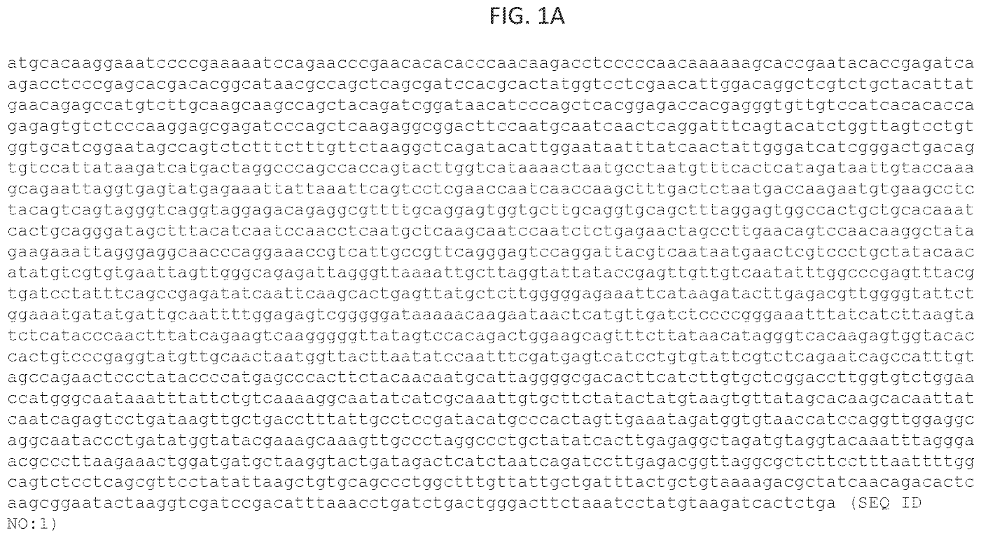

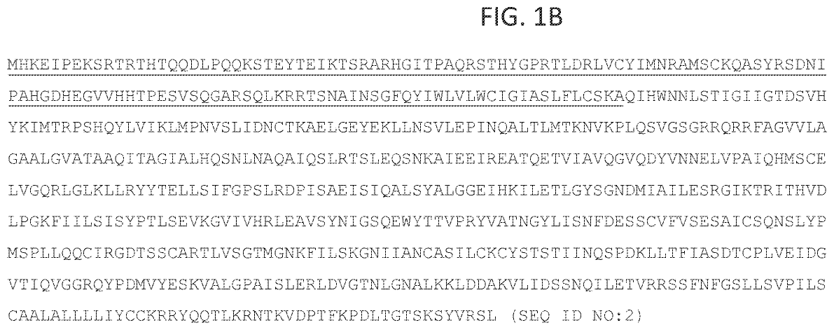

This document provides CDV-F polypeptides. A nucleic acid sequence of a CDV-F open reading frame (SEQ ID NO:1) and an amino acid sequence of an encoded CDV-F polypeptide (SEQ ID NO:2) are set forth in FIGS. 1A and 1B, respectively. Typically, wild-type CDV-F polypeptides contain a signal peptide sequence that is about 135 amino acids in length. The 135 amino acid signal sequence within SEQ ID NO:2 is underlined in FIG. 1B, and is set forth in SEQ ID NO:24.

In some cases, a CDV-F polypeptide can be designed such that virus particles containing the CDV-F polypeptide together with a CDV-H polypeptide exhibit enhanced fusogenic activity. For example, a CDV-F polypeptide can be designed to contain a signal peptide sequence that is no longer than 75 amino acids in length. Truncating the signal peptide sequence of CDV-F polypeptides such that it is no longer than 75 amino acids in length can result in CDV-F polypeptides that, when part of viruses together with CDV-H polypeptides, allow for increased fusogenic activity of the viruses as compared to the level of fusogenic activity exhibited by comparable control viruses containing a CDV-F polypeptide having a full-length wild-type signal peptide sequence (e.g., SEQ ID NO:2)

In some cases, a CDV-F polypeptide provided herein can contain a signal peptide sequence that is from 7 amino acids to 75 amino acids in length. For example, a CDV-F polypeptide provided herein can contain a signal peptide sequence that is from 7 to 75 (e.g., from 7 to 70, from 7 to 65, from 7 to 60, from 7 to 55, from 7 to 50, from 7 to 45, from 7 to 40, from 7 to 35, from 7 to 30, from 7 to 25, from 10 to 75, from 15 to 75, from 20 to 75, from 25 to 75, from 35 to 75, from 45 to 75, from 50 to 75, from 55 to 75, from 65 to 75, from 20 to 60, from 25 to 50, from 30 to 60, or from 30 to 40) amino acids in length. A CDV-F polypeptide provided herein can be produced by truncating a wild-type signal peptide sequence from its N-terminus, from its C-terminus, or from both its N-terminus and C-terminus or by deleting amino acids from in between the N-terminal and C-terminal regions of a wild-type signal peptide sequence. In some cases, a MeV signal peptide sequence can be used for a signal peptide of a CDV-F polypeptide described herein. Examples of signal peptide sequences of CDV-F polypeptides provided herein include, without limitation, those set forth in TABLE 1.

TABLE 1
Examples of signal peptide sequences

| | | SEQ |
|---|---|---|
| Example | | ID |
| # | Sequence | NO: |
| 1 | MNRAMSCKQASYRSDNIPAHGDHEGVV | 3 |
| | HHTPESVSQG | |
| | ARSQLKRRTSNAINSGFQYIWLVLWCI | |
| | GIASLFLCSKA | |
| 2 | MTSNAINSGFQYIWLVLWCIGIASLFL | 4 |
| | CSKA | |
| 3 | MSIMGLKVNVSAIFMAVLLTLQTPTG | 5 |
| 4 | MAINSGSQCTWLVLWCLGIASLFLCSKA | 6 |
| 5 | MAINSGSQCTWLVLWCLGMASLFLCSKA | 7 |
| 6 | MAINSGSQCTWLVLWCFGTASLFLCSKA | 8 |
| 7 | MATSPGPQCTWLVLWCIGIASLFLCSEA | 9 |
| 8 | MAVKSGSQCTWLVLWCIGVASLFLCSKA | 10 |
| 9 | MASNPGSQCTWLVLWCIGIASLFLCSKA | 11 |
| 10 | MATNAGSQYTWLVLWCIGIASLFLCSKA | 12 |
| 11 | MATSSGSQCTWLVLWCIGVASLFLCSKA | 13 |
| 12 | MAINSGSQCTWLVLWCIGVASLFLCSKA | 14 |
| 13 | MAINSGFQYIWLVLWCIGIASLFLCSKA | 15 |

In some cases, a CDV-F polypeptide provided herein can be designed to lack the entire signal peptide sequence. For example, a CDV-F polypeptide provided herein can have an amino acid sequence set forth in SEQ ID NO:2, starting with the amino acid at position 140.

In general, a CDV-F polypeptide provided herein can have any appropriate amino acid sequence, provided that the CDV-F polypeptide does not contain a signal peptide sequence longer than 75 amino acid residues in length. Examples of amino acid sequences of CDV-F polypeptides that can be used as described herein include, without limitation, the amino acid sequences set forth in FIG. 13 of PCT Application Serial No. PCT/US2020/055100.

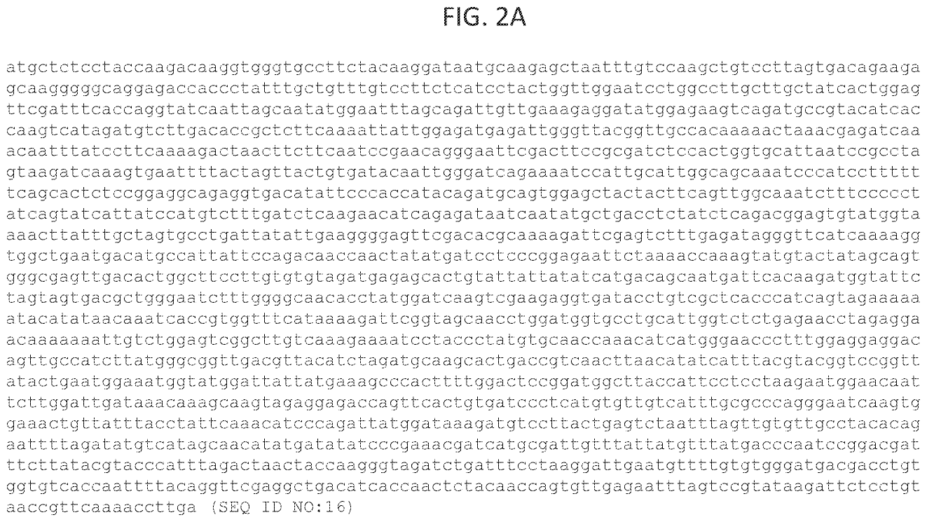

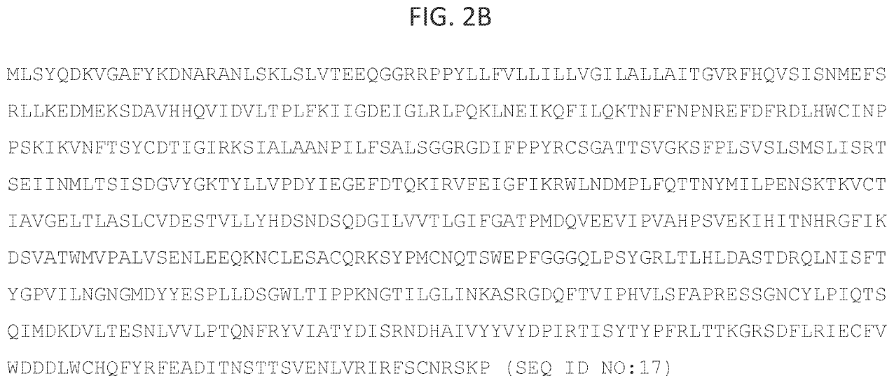

This document also provides CDV-H polypeptides. A nucleic acid sequence of a CDV-H open reading frame (SEQ ID NO:16) and an amino acid sequence of an encoded CDV-H polypeptide (SEQ ID NO:17) are set forth in FIGS. 2A and 2B, respectively.

As described herein, a CDV-H polypeptide can be designed such that viruses containing the CDV-H polypeptide together with a CDV-F polypeptide exhibit altered (e.g., reduced or increased) tropism for SLAMF1 polypeptides and/or NECTIN4 polypeptides as compared to viruses containing wild-type CDV-H polypeptides. For example, a CDV-H polypeptide can be designed to contain a mutation at one or more (e.g., one, two, three, four, five, six, seven, eight, or nine) of amino acid positions 194, 195, 478, 479, 493, 539, 540, 544, and 548. Typically, viruses containing wild-type CDV-H polypeptides (together with CDV-F polypeptides) exhibit tropism for canine slamf1 polypeptides and human or canine NECTIN4 polypeptides, such that the viruses infect SLAMF1-positive cells and NECTIN4-positive cells. Unexpectedly, as described herein, mutating one or more of amino acid positions S194, V195, V478, L479, P493, Y539, D540, T544, and T548 of a CDV-H polypeptide to a different amino acid (e.g., an amino acid from the corresponding position within a MeV H polypeptide) can alter the ability of viruses containing that CDV-H polypeptide (together with a CDV-F polypeptide) to infect SLAMF1-positive cells and/or NECTIN4-positive cells. Examples of CDV-H polypeptides provided herein having altered tropism for SLAMF1 polypeptides and/or NECTIN4 polypeptides include, without limitation, those CDV-H polypeptides having the sequence set forth in SEQ ID NO:17, provided that the CDV-H polypeptide contains a mutation of one or more (e.g., one, two, three, four, five, six, seven, eight, or nine) of S194, V195, V478, L479, P493, Y539, D540, T544, and T548. Examples of amino acid substitutions that can be made at positions 194, 195, 478, 479, 493, 539, 540, 544, and 548 are set forth in TABLE 2. Examples of combinations of the mutations set forth in TABLE 2 that can be used to make a CDV-H polypeptide having altered (e.g., increased or decreased) tropism for human SLAMF1 polypeptides and/or human NECTIN4 polypeptides include, without limitation, those set forth in TABLE 3. For example, a combination of CDV-H mutations to reduce tropism for canine slamf1, human NECTIN4, and canine nectin4 can include V478L, L479D, T544S, and T548D. A combination of CDV-H mutations to reduce tropism for canine slam1, human NECTIN4, and canine nectin4 can include S194I, V195R, V478L, L479D, T544S, and T548D. A combination of CDV-H mutations to increase tropism for human SLAMF1, with reduced tropism for canine slamf1, human NECTIN4, and canine nectin4 can include S194I, V195R, V478L, L479D, D540G, T544S, and T548D.

TABLE 2
Examples of mutations that can be
introduced into a CDV-H polypeptide

| Example # | Position | Amino acid change |
|---|---|---|
| 1 | S194 | I, A, V, L, M, F, Y, W |
| 2 | V195 | R, H, K |
| 3 | V478 | L, I, A, V, M, F, Y, W |
| 4 | L479 | D, E |
| 5 | P493 | S, T, N, Q |
| 6 | Y539 | A, I, V, L, M, F, Y, W |
| 7 | D540 | G, C, P |
| 8 | T544 | S, T, N, Q |
| 9 | T548 | D, E |

TABLE 3
Combinations of mutations from TABLE 2 that
can be included in a CDV-H polypeptide

| Combination # | Combination of mutations from TABLE 2 |
|---|---|
| 1 | 3, 4, 8, and 9 |
| 2 | 1, 2, 3, 4, 8, and 9 |
| 3 | 1, 2, 3, 4, 7, 8, and 9 |

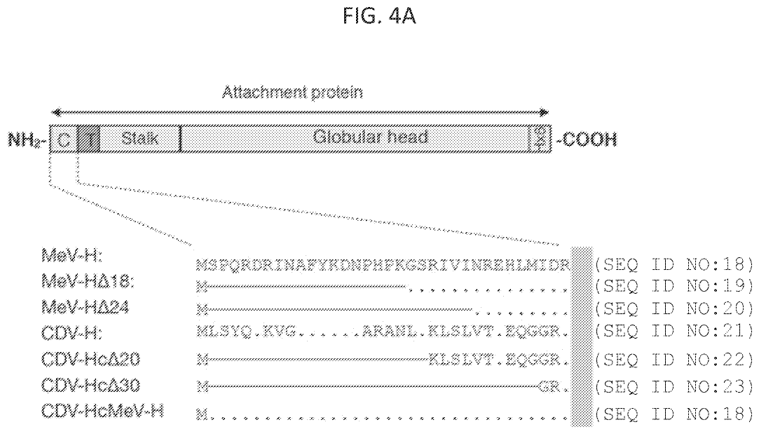

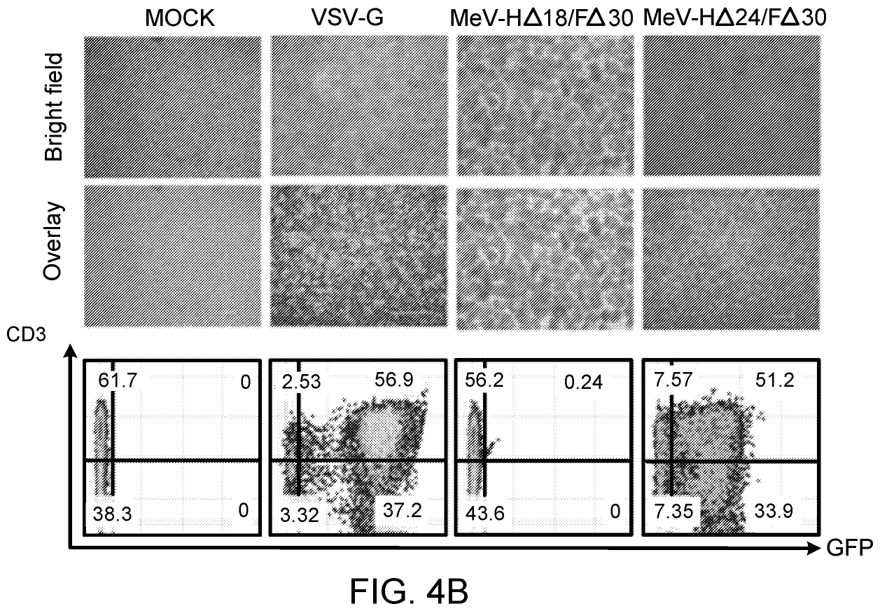

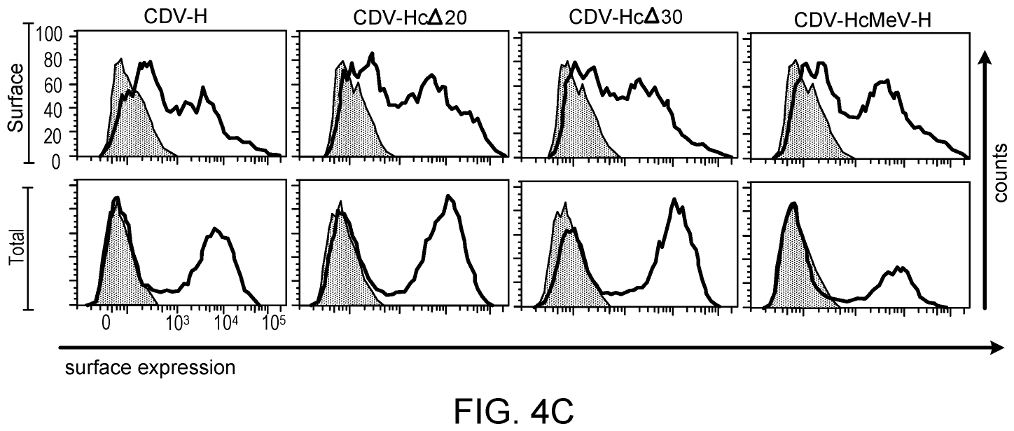

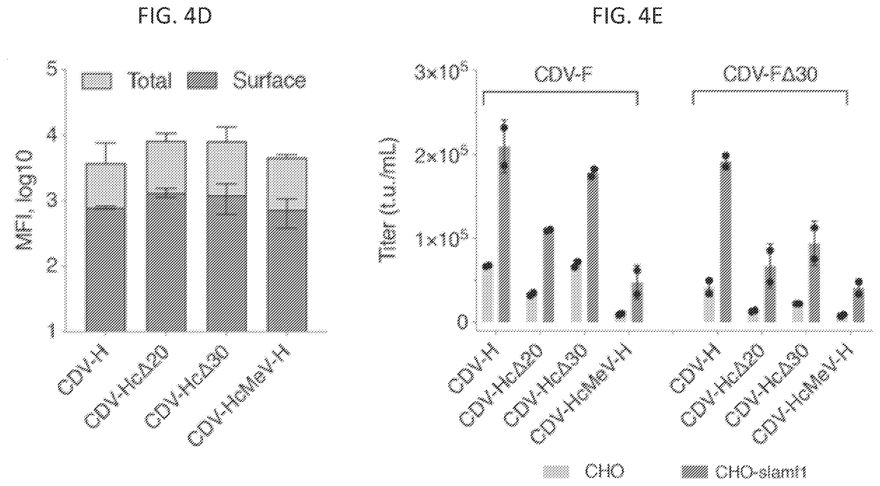

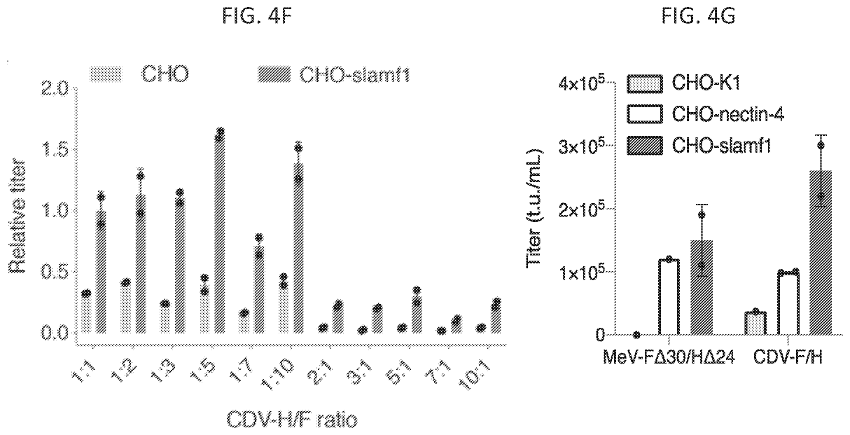

In some cases, a CDV-H polypeptide can be truncated as compared to the CDV-H amino acid sequence set forth in SEQ ID NO:17. The N-terminal 34 amino acids of the sequence set forth in SEQ ID NO:17 comprise a cytoplasmic tail. In some cases, the cytoplasmic tail can be modified to contain a deletion of 20 to 32 amino acids (e.g., a 20 to 23 amino acid deletion, a 23 to 26 amino acid deletion, a 26 to 29 amino acid deletion, a 29 to 32 amino acid deletion, a 22 to 27 amino acid deletion, or a 27 to 32 amino acid deletion). Examples of deletions that can be made within the N-terminal cytoplasmic tail of a CDV-H polypeptide are shown in FIG. 4A.

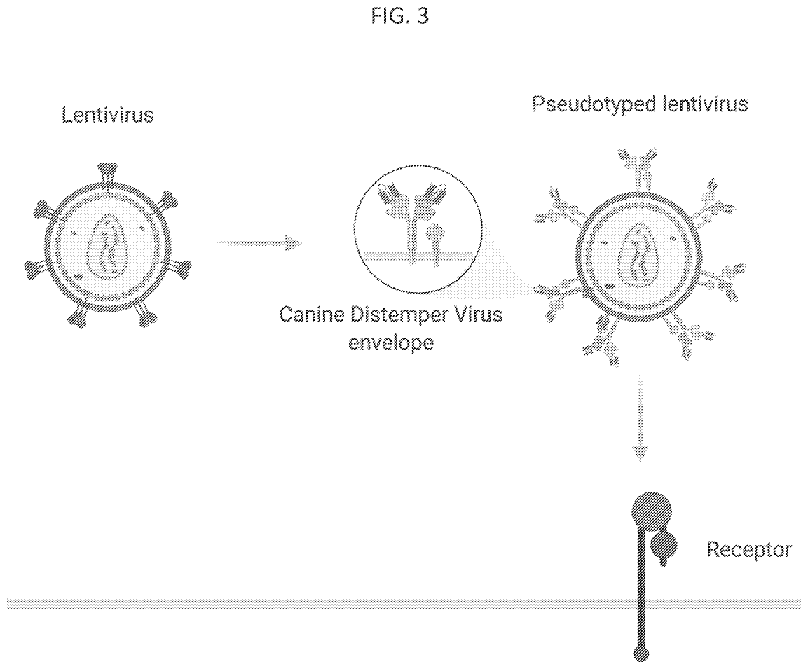

This document also provides virus particles (e.g., pseudotyped LVs; FIG. 3) containing a CDV-H polypeptide provided herein and/or a CDV-F polypeptide provided herein, as well as methods for making viruses (e.g., pseudotyped LVs) containing a CDV-H polypeptide provided herein and/or a CDV-F polypeptide provided herein. For example, a virus (e.g., a pseudotyped LV) can be produced to include (a) a CDV-H polypeptide provided herein and a wild-type CDV-F polypeptide, or (b) a CDV-H polypeptide provided herein and a CDV-F polypeptide described herein. In some cases, a virus (e.g., a pseudotyped LV) can be produced to include a CDV-H polypeptide having mutations 1, 2, 3, 4, 8, and 9 from TABLE 2, and a wild type CDV-F polypeptide or a CDV-F polypeptide provided herein produced by truncating a wild-type signal peptide sequence at its N-terminus, at its C-terminus, or at both its N-terminus and C-terminus, or by deleting amino acids from in between the N-terminal and C-terminal regions of a wild-type signal peptide sequence. In some cases, a virus (e.g., a pseudotyped LV) can be produced to include a CDV-H polypeptide having mutation 7 from TABLE 2, and a wild type CDV-F polypeptide or a CDV-F polypeptide having or a CDV-F polypeptide provided herein produced by truncating a wild-type signal peptide sequence at its N-terminus, at its C-terminus, or at both its N-terminus and C-terminus, or by deleting amino acids from in between the N-terminal and C-terminal regions of a wild-type signal peptide sequence. In some cases, a virus (e.g., a pseudotyped LV) can be produced to include a CDV-H polypeptide having mutations 1, 2, 3, 4, 7, 8, and 9 from TABLE 2, and a wild type CDV-F polypeptide or a CDV-F polypeptide having or a CDV-F polypeptide provided herein produced by truncating a wild-type signal peptide sequence at its N-terminus, at its C-terminus, or at both its N-terminus and C-terminus, or by deleting amino acids from in between the N-terminal and C-terminal regions of a wild-type signal peptide sequence.

This document also provides nucleic acid molecules encoding a CDV-H polypeptide provided herein and/or nucleic acid molecules encoding a CDV-F polypeptide provided herein. For example, a nucleic acid molecule (e.g., a vector) can be designed to encode a CDV-H polypeptide provided herein and/or a CDV-F polypeptide provided herein.

This document provides methods and materials related to LVs. For example, this document provides pseudotyped LVs, methods for making pseudotyped LVs, and methods for using pseudotyped LVs to treat cancer or infectious diseases.

As described herein, a pseudotyped LV can be produced to contain CDV-H and/or F polypeptides on its surface, without containing any CDV nucleic acid sequences. Methods for generating pseudotyped LVs can include transducing LVs into producer cells that express nucleic acids encoding a CDV-H polypeptide (e.g., a CDV-H polypeptide provided herein) and a CDV-F polypeptide (e.g., a wild type CDV-F polypeptide or a CDV-F polypeptide provided herein). In such cases, the LVs can replicate in the producer cells, and the newly produced LV particles can include CDV-H and F polypeptides on their outer surfaces.

Any appropriate cells can be used as producer cells for generating pseudotyped LVs. Non-limiting examples of cells that can be used as producer cells include HEK 293 cells, HEK 293T cells, RDF21 HeLa cells, PT67 cells, and Phoenix-GP cells. To generate producer cells, one or more nucleic acid molecules encoding a CDV-H polypeptide and/or a CDV-F polypeptide can be introduced into cells of a selected type (e.g., HEK 293T cells), and the cells can be cultured under conditions suitable for expression of the introduced CDV polypeptides. The CDV-H polypeptide coding sequence and/or the CDV-F polypeptide coding sequence can be stably integrated into the producer cell genome, or the CDV-H and/or F coding sequence(s) can be transiently expressed by the producer cell.

Any appropriate nucleic acid encoding a CDV-F polypeptide can be introduced into cells that are to be used as producer cells for pseudotyped LVs. For example, nucleic acid encoding a wild-type CDV-F polypeptide or a CDV-F polypeptide provided herein can be introduced into cells that are to be used as producer cells for pseudotyped LV.

Any appropriate nucleic acid encoding a CDV-H polypeptide can be introduced into cells that are to be used as producer cells for pseudotyped LVs. For example, nucleic acid encoding a wild-type H polypeptide or a H polypeptide provided herein can be introduced into cells that are to be used as producer cells for pseudotyped LVs. In some cases, nucleic acid encoding a CDV-H polypeptide that has altered (e.g., reduced or increased) specificity for SLAMF1 and/or NECTIN4 can be introduced into cells that are to be used as producer cells for pseudo typed LVs. For example, nucleic acid encoding a CDV-H polypeptide having one or more mutations set forth in TABLE 2 can be introduced into cells that are to be used as producer cells for pseudotyped LVs.

In some cases, a pseudotyped LV can contain a CDV-H polypeptide and/or a CDV-F polypeptide designed to have a preselected tropism. For example, CDV-F and/or H polypeptides having knocked out specificity for SLAMF1 and/or NECTIN4 can be used such that a scFv or polypeptide ligand can be attached to, for example, the C-terminus of the CDV-H polypeptide. In such cases, scFv or polypeptide ligand can determine the tropism of a pseudotyped LV. Examples of scFvs that can be used to direct pseudotyped LVs to cellular receptors (e.g., tumor associated cellular receptors) include, without limitation, anti-EGFR, anti-VEGFR, anti-CD46, anti-αFR, anti-PSMA, anti-HER-2, anti-CD19, anti-CD20, anti-CD4, anti-CD8, anti-CD3, anti-CD34, anti-CD117 (c-Kit), anti-EpCAM, anti-CD33, anti-CD133, anti-CD135 (Flt3), and anti-CD38 scFvs. Examples of polypeptide ligands that can be used to direct pseudotyped LVs include, without limitation, EGF ligand, urokinase plasminogen activator uPA polypeptides, cytokines such as IL-13, single chain T cell receptors (scTCRs), echistatin polypeptides, integrin binding polypeptides, stem cell factor (SCF), Flt3 ligand, affibodies, and DARPins.

The nucleic acid sequences of a pseudotyped LV provided herein that include LV gag, pol, and env sequences can, in some cases, be from a NY5/BRU strain as set forth in GENBANK® Accession No. AF324493.2.

In some cases, the LV nucleic acid molecule of a pseudotyped LV provided herein can encode an IFN polypeptide, a fluorescent polypeptide (e.g., a GFP polypeptide), a NIS polypeptide, a therapeutic polypeptide, an innate immunity antagonizing polypeptide, a tumor antigen, a toxin, a chimeric antigen receptor (CAR) polypeptide, or a combination thereof.

Nucleic acid encoding an IFN polypeptide can be positioned in the nef or gag frame, for example. Such a position can allow the viruses to express an amount of IFN polypeptide that is effective to activate anti-viral innate immune responses in non-cancerous tissues, and thus alleviate potential viral toxicity, without impeding efficient viral replication in cancer cells.

Any appropriate nucleic acid encoding an IFN polypeptide can be inserted into the genome of a LV. For example, nucleic acid encoding an IFN beta polypeptide can be inserted into the genome of a LV. Examples of nucleic acid encoding IFN beta polypeptides that can be inserted into the genome of a LV include, without limitation, nucleic acid encoding a human IFN beta polypeptide of the nucleic acid sequence set forth in GENBANK® Accession No. NM_002176.2 (GI No. 50593016), nucleic acid encoding a mouse IFN beta polypeptide of the nucleic acid sequence set forth in GENBANK® Accession Nos. NM_010510.1 (GI No. 6754303), BC119395.1 (GI No. 111601321), or BC119397.1 (GI No. 111601034), and nucleic acid encoding a rat IFN beta polypeptide of the nucleic acid sequence set forth in GENBANK® Accession No. NM_019127.1 (GI No. 9506800).

Nucleic acid encoding a NIS polypeptide can be positioned in the env open reading frame, for example. Such a position can allow the LVs to express an amount of NIS polypeptide that (a) is effective to allow selective accumulation of iodide in infected cells, thereby allowing both imaging of viral distribution using radioisotopes and radiotherapy targeted to infected cancer cells, and (b) is not so high as to be toxic to infected cells.

Any appropriate nucleic acid encoding a NIS polypeptide can be inserted into the genome of a LV. For example, nucleic acid encoding a human NIS polypeptide can be inserted into the genome of a LV. Examples of nucleic acid encoding NIS polypeptides that can be inserted into the genome of a LV include, without limitation, nucleic acid encoding a human NIS polypeptide of the nucleic acid sequence set forth in GENBANK® Accession Nos. NM_000453.2 (GI No.164663746), BC105049.1 (GI No. 85397913), or BC105047.1 (GI No. 85397519), nucleic acid encoding a mouse MS polypeptide of the nucleic acid sequence set forth in GENBANK® Accession Nos. NM_053248.2 (GI No. 162138896), AF380353.1 (GI No. 14290144), or AF235001.1 (GI No. 12642413), nucleic acid encoding a chimpanzee NIS polypeptide of the nucleic acid sequence set forth in GENBANK® Accession No. XM_524154 (GI No. 114676080), nucleic acid encoding a dog NIS polypeptide of the nucleic acid sequence set forth in GENBANK® Accession No. XM_541946 (GI No. 73986161), nucleic acid encoding a cow NIS polypeptide of the nucleic acid sequence set forth in GENBANK® Accession No. XM_581578 (GI No. 297466916), nucleic acid encoding a pig NIS polypeptide of the nucleic acid sequence set forth in GENBANK® Accession No. NM_214410 (GI No. 47523871), and nucleic acid encoding a rat NIS polypeptide of the nucleic acid sequence set forth in GENBANK® Accession No. NM_052983 (GI No. 158138504).

Nucleic acid encoding an toxin polypeptide can be positioned in the nef or gag frame, or in place of the env open reading frame. Such a position can allow the viruses to express an amount of toxin polypeptide that is effective to kill a target cell (e.g., a cancer cell).

Any appropriate nucleic acid encoding a toxin polypeptide can be inserted into the genome of a LV. For example, nucleic acid encoding the prodrug convertase purine nucleotide phosphorylase (PNP), neutrophil-activating protein of Helicobacter pylori (HP-NAP), co-chaperonin GroEs, human granulocyte-macrophage colony stimulating factor (GM-CSF), Escherichia coli cytosine deaminase, or human herpesvirus thymidine kinase can be inserted into the genome of a LV. Examples of nucleic acid molecules encoding toxin polypeptides that can be inserted into the genome of a LV include, without limitation, nucleic acid encoding the prodrug convertase PNP set forth in GENBANK® Accession No. M60917.2, nucleic acid encoding the HP-NAP polypeptide set forth in GENBANK® Accession No. WO_000846461.1, nucleic acid encoding the co-chaperonin GroEs set forth in GENBANK® Accession No. CP003904.1, nucleic acid encoding the human GM-CSF polypeptide set forth in GENBANK® Accession No. M11220.1), nucleic acid encoding Escherichia coli cytosine deaminase polypeptide set forth in (GENBANK® Accession No. AR014075.1), and nucleic acid encoding the human herpesvirus thymidine kinase polypeptide set forth in GENBANK® Accession No. AB009254.2.

Nucleic acid encoding a CAR polypeptide that combines antigen-binding function and T-cell activating function can be positioned in place of the env open reading frame. Such a position can allow the viruses to express an amount of CAR polypeptide that is effective to give T cells the ability to target a specific polypeptide. A CAR polypeptide coding sequence can be contained within a pseudotyped LV with tropism for, without limitation, CD3, CD4, or CD8. A CAR polypeptide can be designed to bind any appropriate antigen (e.g., CD19, CD20, CD22, or B cell maturation antigen).

In some cases, the virus nucleic acid in a pseudotyped LV can be disarmed, such that it cannot replicate within a target cell (e.g., a T cell or a cancer cell). Using pseudotyped LVs with disarmed (replication incompetent) LV nucleic acid can prevent the LV from propagating in target cells and subsequently infecting cells other than those that were originally targeted based on the tropism of the pseudotyped LV.

Any appropriate method can be used to insert nucleic acid (e.g., nucleic acid encoding an IFN polypeptide and/or nucleic acid encoding a NIS polypeptide and/or nucleic acid encoding a CAR polypeptide and/or nucleic acid encoding a toxin) into the genome of a LV. For example, methods described elsewhere (Naamati et al., Elife 8:e41431, 2019; and Uhlig et al., J Virol, 89(17):9044-9060, 2015) can be used to insert nucleic acid into the genome of a LV. Any appropriate method can be used to identify LVs containing a nucleic acid molecule described herein. Such methods include, without limitation, PCR and nucleic acid hybridization techniques such as Northern and Southern analysis. In some cases, immunohistochemistry and biochemical techniques can be used to determine if a LV contains a particular nucleic acid molecule by detecting the expression of a polypeptide encoded by that particular nucleic acid molecule.

The term “nucleic acid” as used herein encompasses both RNA (e.g., viral RNA) and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

This document also provides method for treating cancer (e.g., to reduce tumor size, inhibit tumor growth, or reduce the number of viable tumor cells), methods for inducing host immunity against cancer, and methods for treating an infectious disease such as an HIV or measles infection. For example, a pseudotyped virus (e.g., a pseudotyped retrovirus, such as LV) provided herein can be administered to a mammal having cancer to reduce tumor size, to inhibit cancer cell or tumor growth, to reduce the number of viable cancer cells within the mammal, and/or to induce host immunogenic responses against a tumor. A pseudotyped virus (e.g., a pseudotyped LV) provided herein can be propagated in host producer cells to yield a sufficient number of copies of that virus for use in a method provided herein. A viral titer typically is assayed by inoculating cells (e.g., Vero cells) in culture.

Pseudotyped viruses (e.g., pseudotyped LVs) provided herein can be administered to a cancer patient by, for example, direct injection into a group of cancer cells (e.g., a tumor) or intravenous delivery to cancer cells. A pseudotyped virus (e.g., a pseudotyped LV) provided herein can be used to treat different types of cancer including, without limitation, myeloma (e.g., multiple myeloma), melanoma, glioma, lymphoma, mesothelioma, and cancers of the lung, brain, stomach, colon, rectum, kidney, prostate, ovary, breast, pancreas, liver, and head and neck.

Pseudotyped viruses (e.g., pseudotyped LVs) provided herein can be administered to a patient in a biologically compatible solution or a pharmaceutically acceptable delivery vehicle, by administration either directly into a group of cancer cells (e.g., intratumorally) or systemically (e.g., intravenously). Suitable pharmaceutical formulations depend in part upon the use and the route of entry, e.g., transdermal or by injection. Such forms should not prevent the composition or formulation from reaching a target cell (i.e., a cell to which the virus is desired to be delivered to) or from exerting its effect. For example, pharmacological compositions injected into the blood stream should be soluble.

While dosages administered will vary from patient to patient (e.g., depending upon the size of a tumor), an effective dose can be determined by setting as a lower limit the concentration of virus proven to be safe and escalating to higher doses of up to 10¹² pfu, while monitoring for a reduction in cancer cell growth along with the presence of any deleterious side effects. A therapeutically effective dose typically provides at least a 10% reduction in the number of cancer cells or in tumor size. Escalating dose studies can be used to obtain a desired effect for a given viral treatment (see, e.g., Nies and Spielberg, “Principles of Therapeutics,” In Goodman & Gilman's The Pharmacological Basis of Therapeutics, eds. Hardman, et al., McGraw-Hill, NY, 1996, pp 43-62).

Pseudotyped viruses (e.g., pseudotyped LVs) provided herein can be delivered in a dose ranging from, for example, about 10³ transducing units per kg (TU/kg) to about 10¹² TU/kg (e.g., about 10⁵ TU/kg to about 10¹² TU/kg, about 10⁶ TU/kg to about 10¹¹ TU/kg, or about 10⁶ TU/kg to about 10¹⁰ TU/kg). A therapeutically effective dose can be provided in repeated doses. Repeat dosing can be appropriate in cases in which observations of clinical symptoms or tumor size or monitoring assays indicate either that a group of cancer cells or tumor has stopped shrinking or that the degree of viral activity is declining while the tumor is still present. Repeat doses can be administered by the same route as initially used or by another route. A therapeutically effective dose can be delivered in several discrete doses (e.g., days or weeks apart) and in one embodiment, one to about twelve doses are provided. Alternatively, a therapeutically effective dose of pseudotyped viruses (e.g., pseudotyped LVs) provided herein can be delivered by a sustained release formulation. In some cases, a pseudotyped virus (e.g., a pseudotyped LV) provided herein can be delivered in combination with pharmacological agents that facilitate viral replication and spread within cancer cells or agents that protect non-cancer cells from viral toxicity. Examples of such agents are described elsewhere (Alvarez-Breckenridge et al., Chem. Rev., 109(7):3125-40 (2009)).

Pseudotyped viruses (e.g., pseudotyped LVs) provided herein can be administered using a device for providing sustained release. A formulation for sustained release of pseudotyped viruses (e.g., LVs) provided herein can include, for example, a polymeric excipient (e.g., a swellable or non-swellable gel, or collagen). A therapeutically effective dose of pseudotyped viruses (e.g., pseudotyped LVs) provided herein can be provided within a polymeric excipient, wherein the excipient/virus composition is implanted at a site of cancer cells (e.g., in proximity to or within a tumor). The action of body fluids gradually dissolves the excipient and continuously releases the effective dose of virus over a period of time. Alternatively, a sustained release device can contain a series of alternating active and spacer layers. Each active layer of such a device typically contains a dose of virus embedded in excipient, while each spacer layer contains only excipient or low concentrations of virus (i.e., lower than the effective dose). As each successive layer of the device dissolves, pulsed doses of virus are delivered. The size/formulation of the spacer layers determines the time interval between doses and is optimized according to the therapeutic regimen being used.

In some cases, pseudotyped viruses (e.g., pseudotyped LVs) provided herein can be directly administered. For example, a virus can be injected directly into a tumor (e.g., a breast cancer tumor) that is palpable through the skin. Ultrasound guidance also can be used in such a method. Alternatively, direct administration of a virus can be achieved via a catheter line or other medical access device, and can be used in conjunction with an imaging system to localize a group of cancer cells. By this method, an implantable dosing device typically is placed in proximity to a group of cancer cells using a guidewire inserted into the medical access device. An effective dose of a pseudotyped virus (e.g., a pseudotyped LV) provided herein can be directly administered to a group of cancer cells that is visible in an exposed surgical field.

In some cases, pseudotyped viruses (e.g., pseudotyped LVs) provided herein can be delivered systemically. For example, systemic delivery can be achieved intravenously via injection or via an intravenous delivery device designed for administration of multiple doses of a medicament. Such devices include, but are not limited to, winged infusion needles, peripheral intravenous catheters, midline catheters, peripherally inserted central catheters, and surgically placed catheters or ports.

The course of therapy with a pseudotyped virus (e.g., a pseudotyped LV) provided herein can be monitored by evaluating changes in clinical symptoms or by direct monitoring of the number of cancer cells or size of a tumor. For a solid tumor, the effectiveness of virus treatment can be assessed by measuring the size or weight of the tumor before and after treatment. Tumor size can be measured either directly (e.g., using calipers), or by using imaging techniques (e.g., X-ray, magnetic resonance imaging, or computerized tomography) or from the assessment of non-imaging optical data (e.g., spectral data). For a group of cancer cells (e.g., leukemia cells), the effectiveness of viral treatment can be determined by measuring the absolute number of leukemia cells in the circulation of a patient before and after treatment. The effectiveness of viral treatment also can be assessed by monitoring the levels of a cancer specific antigen. Cancer specific antigens include, for example, carcinoembryonic antigen (CEA), prostate specific antigen (PSA), prostatic acid phosphatase (PAP), CA 125, alpha-fetoprotein (AFP), carbohydrate antigen 15-3, and carbohydrate antigen 19-4.

The invention will be further described in the following examples, which do not limit the scope of the invention described in the claims.

### EXAMPLE S

### Example 1—Methods and Materials

Cell lines: HEK293T cells were maintained in Dulbecco's modified Eagle's medium (DMEM, Cat. #SH30022.01; GE Healthcare Life, Pittsburg, PA). Jurkat cells were cultured in RPMI 1640—10% FBS. Chinese hamster ovary (CHO), CHO-CD46 (Nakamura et al., Nat Biotechnol 2004, 22(3):331-336), CHO-hSLAMF1 (Tatsuo et al., Nature 2000, 406(6798):893-897), CHO-dogSLAMF1 (Seki et al., J Virol 2003, 77(18):9943-9950), CHO-NECTIN4 (Liu et al., J Virol 2014, 88(4):2195-2204), CHO-αHIS (Nakamura et al., Nat Biotechnol 2005, 23(2):209-214), CHO-CD38 (Peng et al., Blood 2003, 101(7):2557-2562), and CHO-EpCAM (Munch et al., Nature Commun 2015, 6:6246) cells were grown in RPMI 1640 medium supplemented with 10% FBS as described elsewhere. CHO cells constitutively expressing the dog nectin4 molecule (CHO-nectin4) were obtained from Imanis Life Science (Rochester, MN). All cells were additionally supplemented with 1% Penicillin/Streptomycin (Cat. #30-002-CI; Corning Inc, Corning, NY) and 10 mM N-2-hydroxyethylpiperazine-N-2-ethane sulfonic acid (HEPES, Cat. #15630-080; ThermoFisher Scientific, Waltham, MA), and were incubated at 37° C. in 5% CO₂ with saturating humidity.

Plasmid constructs: To generate CDV SPA.Madrid/22458/16 expression plasmids, total RNA was extracted from CDV SPA.Madrid/22458/16 isolate-infected Vero/dog SLAMF1 cells (passage 1) using the RNeasy Mini Kit (Qiagen, Hilden, Germany). The CDV-hemagglutinin (H) and CDV-fusion (F) genes were reverse transcribed with SuperScript III Reverse Transcriptase (Cat. #11752050, ThermoFisher Scientific) and amplified by PCR with the following primers:

CDVH7050(+):
(SEQ ID NO: 25)
5′-AGAAAACTTAGGGCTCAGGTAGTCC-3′
CDVH8949(−):
(SEQ ID NO: 26)
5′-TCGTCTGTAAGGGATTTCTCACC-3′
CDVF4857(+):
(SEQ ID NO: 27)
5′-AGGACATAGCAAGCCAACAGG-3′
CDVH7050(−):
(SEQ ID NO: 28)
5′-GGACTACCTGAGCCCTAAGTTTTCT-3′

PCR products were sequenced directly by Sanger sequencing (Genewiz; Plainfield, NJ) and cloned into the pJET1.2 vector (ThermoFisher Scientific). Next, the CDV-H open reading frame was PCR amplified with a forward primer (5′-CCGGTAGTTAATTAA AACTTAGGGTGCAAGATCATCGATAATGCTCTCCTACCAAGATAAGGTG-3; SEQ ID NO:29) and a reverse primer (5′-CTATTTCACACTAGTGGGTATGCCTGATGTCTG GGTGACATCATGTGATTGGTTCACTAGCAGCCTCAAGGTTTTGAACGGTTACAG GAG-3; SEQ ID NO:30) and cloned into a PacI and SpeI-restricted (New England Biolabs, Ipswich, MA) pCG vector (Cathomen et al., J Virol 1998, 72(2):1224-1234) using an InFusion HD kit (Takara; Shinagawa, Tokyo, Japan). The primers contained the PacI and SpeI restriction sites (underlined) as well as coding sequence for the untranslated region of MeV-H (italics). Similarly, the CDV-F open reading frame (amino acid residues 136-662) was cloned into the HpaI/SpeI-restricted pCG-CDV-F plasmid (von Messling et al., J Virol 2001, 75(14):6418-6427). The resulting plasmid pCG-CDV-F SPA.Madrid/22458/16 contained coding sequences for the MeV-F untranslated region and signal peptide.

To generate the shuttle vector pTN-CDV-H-IdeZ coding for CDV-H protein followed by the IgG1 hinge sequence instead of original factor Xa cleavage site (IEGR) in the plasmid pTNH6 (Nakamura et al. 2005, supra), a Eam1105I restriction site in the ampicillin was first eliminated by site-directed mutagenesis (QuickChange Site-Directed Mutagenesis Kit; Agilent Technologies, Santa Clara CA) and the CDV-H coding sequence with the C-terminus linker was then introduced by recombination of gBlock fragments (IDT; Newark, NJ) using InFusion cloning methods (InFusion HD kit; Takara BIO, Kyoto, Japan). The DARPin Ac1 targeting domain was synthesized and introduced into the Eam11051/NotI sites on the pTN-CDV-H-IdeZ vector. Truncated cytoplasmic tails were introduced by PCR amplification and insertion of fragments into the pTN-CDV-H-IdeZ and pCG-F vectors. Mutations to ablate the natural tropism for cognate receptors were introduced by site-directed mutagenesis.

Swapping of the cytoplasmic tail of the CDV H protein with the heterologous tail from measles virus H protein was achieved by PCR amplification of CDV-H using the following primers: 5′-CCCGGTAGTTAATTAAAACTTAGGGTGCAAGATCATCG ATAATGTCACCACAACGAGACCGGATAAATGCCTTCTACAAAGATAACCCCC ATCCCAAGGGAAGTAGGATAGTCATTAACAGAGAACATCTTATGATTGATAG ACCACCCTATTTGCTGTTTGTCCTTC-3′ (SEQ ID NO:31) and 5′-GCGAAGACTGA CGGTCCCCCCAGGAGTTCAGGTGCTGGGCACGGTGGGCAAGGTTTTGAACGG TTACAGGAGAATC-3′ (SEQ ID NO:32). Fragments were cloned back into the PacI and Eam1105I restricted pTN-CDV-H-IdeZ plasmid using the InFusion HD kit (Takara).

Vector production and transduction experiments: Wild-type CDV glycoproteins and VSV-G pseudotyped lentiviral particles (LV) were generated by transfection of HEK293T cells using TransIT-LT1 (Minis Bio; Madison, WI). For experiments using luciferase, VSV-G-LV were produced as described elsewhere (Crawford et al., Viruses 2020, 12(5):513). For codisplay of CDV F/H-LV, the VSV-G encoding plasmid was substituted with pCG-CDV-F and pCG-CDV-H plasmids without varying the amount of total DNA. For experiments using GFP, pLV-SFF-eGFP-PGK-Puro (Imanis Life Sciences; Rochester MN) and psPAX2 (Addgene, Cat. #12260) were used at a ratio of 1.3:1:4.7:4.9 between packaging plasmid, transgene plasmid, CDV-F plasmid, and CDV-H plasmid. 24 hours after transfection, the medium was replaced to complete medium was antibiotics. Cell supernatants containing LV were collected 48 hours post-transfection and passed through a 0.45 μm pore size filter.

For transduction, 3×10⁴ CHO cells and derivates were seeded into 96-well plates overnight. Cells were transduced with 5-fold dilutions of supernatant-containing LVs, and the inoculum was replaced the next day with fresh medium. Cells were analyzed for luciferase expression 72 hours after infection using Bright-Glo Luciferase Assay System (Cat. #E2610, Promega; Madison, WI) and an Infinite M200Pro microplate reader (Tecan) with no attenuation and a luminescence integration time of 1 second. For titration, GFP-positive cells were determined by flow cytometry using a ZE5 cell analyzer (Bio-Rad; Hercules, CA) and transducing units per milliliter (t.u./mL) were calculated from dilutions giving between 1-20% of GFP-positive cells.

Expression analysis of Morbillivirus attachment proteins: Transfected HEK293 cells were washed with Dulbecco's Phosphate Buffered Saline (DPBS, Cat. #MT-21-0312-CVRF, Corning, Inc.) and detached with TrypLE Express Enzyme (ThermoFisher). Cells were fixed with IC fixation buffer (Cat. #00-8222, ThermoFisher) followed by incubation with phycoerythrin-conjugated anti-6×HIS-tag monoclonal antibody (Cat. #130-120-787, Miltenyi Biotec; Bergisch Gladbah, Germany). For analysis of total protein expression, cells were permeabilized with 1×permeabilization buffer (Cat. #00-8333, ThermoFisher). After three washes with FACS buffer (phosphate-buffered saline (PBS) containing 1%-FBS, 5 mM ethylenediaminetetraacetic acid (EDTA), 1% sodium azide), cells were resuspended in FACS buffer containing 1% paraformaldehyde (PFA) and analyzed using FlowJo Software for Mac (version 10.6.0, Becton Dickinson Co.).

SDS-PAGE and immunoblotting: Supernatant-containing LVs were precipitated with PEG-it (SBI System Bioscience; Palo Alto, CA) and 0.5 μg of total protein were fractionated into 4-12% Bis-Tris polyacrylamide gel, and transferred to polyvinylidene fluoride membranes. Blots were analyzed with 0.2 μg/mL, anti-HIS antibody (Cat. #A01857-40, GenScript; Piscataway, NJ) or 1:1000 anti-p24 antibody (Sino Cat. #11695-RP02; Sino Biological, Beijing, China) and probed with horseradish peroxidase conjugated secondary antibody (Cat.# R1006, Kindle Biosciences; Greenwich, CT). The blots were incubated with Ultra Digital-ECL Substrate (Kindle Biosciences) and analyzed with a KwikQuant imager (Kindle Biosciences).

Structural Modeling: A model of the CDV-H with >90% confidence was generated with the program Phyre2 (Kelley et al., Nat Protoc 2015, 10(6):845-858). The MeV-H/SLAMF1 crystallographic costructure (PDB 3ALZ) was superimposed to the modelled CDV-H structure to identify putative key residues important for human SLAMF1 interaction. The structures were analyzed and manipulated with MacPyMOL software v1.7.6.3 (pymol.org).

### Example 2—Results

Pseudotyped LVs were generated with several H polypeptides. Various truncated versions of MeV and CDV-H polypeptides displaying a 6×H tag at the C-terminus were generated (FIG. 4A), as was a CDV-H chimeric polypeptide containing a MeV-H cytoplasmic domain (CDV-HcMeV-H, FIG. 4A). Jurkat cells were transduced with LVs displaying various H and F polypeptides, and the percentage of GFP positive cells within the CD3 population was measured by flow cytometry four days after transduction (FIG. 4B). These studies demonstrated LV pseudotyping with and without truncation of the MeV-F/H cytoplasmic tail. In subsequent work, HEK-293T cells were transiently transfected with expression plasmids encoding CDV-H truncation variants or the CDV-HcMeV-H chimeric polypeptide, and flow cytometry was used to measure surface and total expression (FIG. 4C). Mean fluorescence intensities for the positively stained cell population indicated no significant difference between native and variant CDV-H polypeptides (FIG. 4D). Screening titers of unconcentrated LV produced using various CDV-F/H combinations showed that truncation of the CDV-H polypeptide by 20 or 30 amino acids reduced the titer in CHO cells expressing canine slamf1, but that the larger truncation had less of an effect (FIG. 4E). In addition, F/H combinations with a truncated F polypeptide displayed somewhat lower titers than combinations with a full length F polypeptide, although both still permitted infection of CHO-slamf1 cells. Studies using unconcentrated LVs produced by varying the ratio of CDV-F/H-expression plasmids showed higher titers in target CHO-slamf1 cells when the CDV-F expression plasmid was present in an amount equal to or greater than the CDV-H expression plasmid (FIG. 4F). Additional studies using MeV-HΔ24/FΔ30 and CDV-F/H-displaying LVs with a F/H ratio of 1:1 revealed that LVs displaying either truncated or untruncated H and F polypeptides transduced CHO cells expressing canine nectin-4 or slamf1 (FIG. 4G).

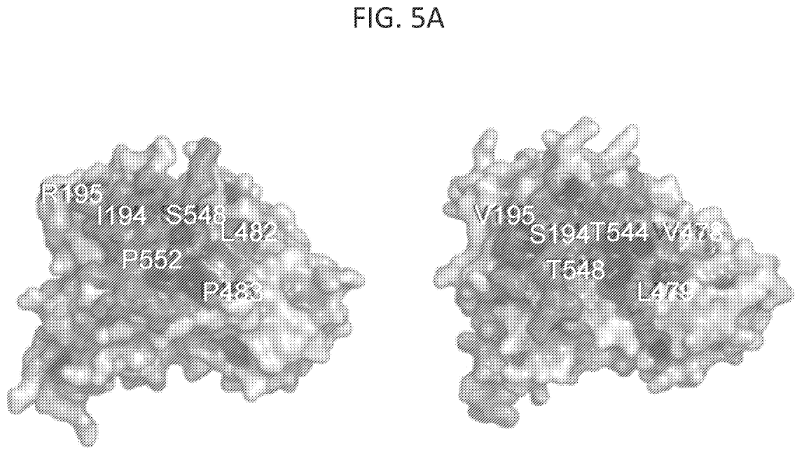

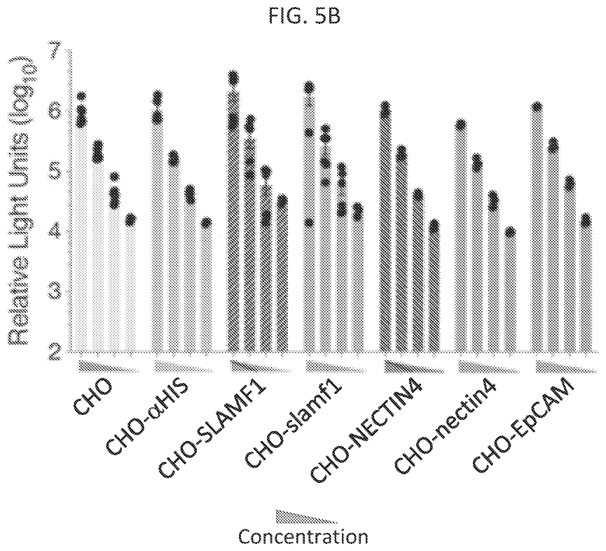

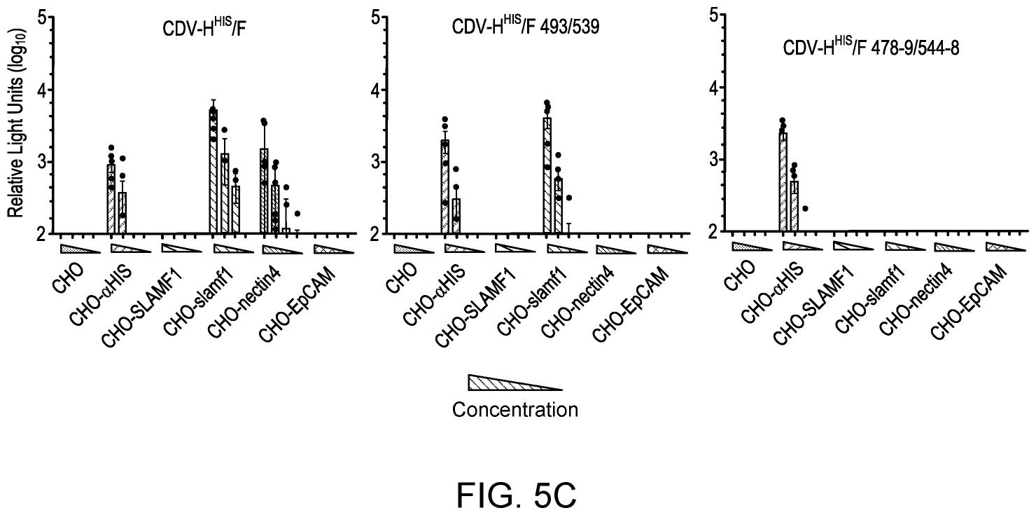

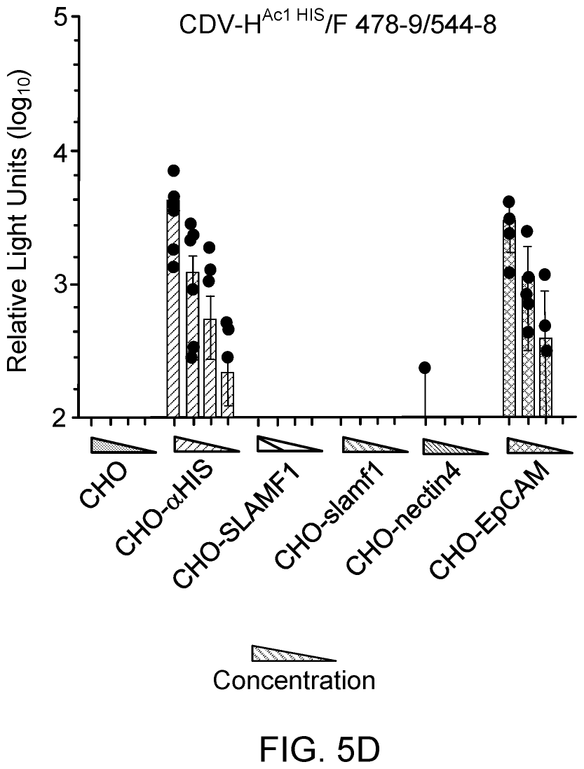

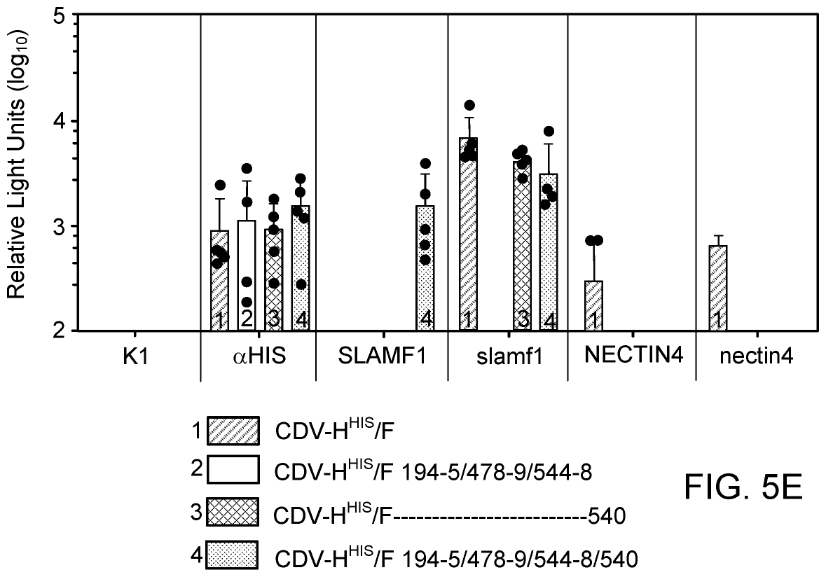

The F/H complex of CDV can mediate fusion via interaction of the H polypeptide with human receptors NECTIN4 and SLAMF1. Mutations were introduced to alter the receptor tropisms, and LV particles displaying various mutated CDV-F/H glycoproteins were tested for retargeting. The structure of CDV-H in complex with SLAMF1 was modeled (FIG. 5A, right) using Pyre2, and various CDV-H residues were substituted to their MeV-H counterparts for retargeting purposes, yielding the following substitutions in various combinations: S194I, V195R, V478L, L479D, T544S, T548D, P493S, Y539A, and D540G. FIG. 5B indicates levels of transduction of VSV-G-displaying LVs in stably-expressing CHO cell lines. Starting at a 1:2 dilution, five-fold dilutions of supernatant-containing LV particles were used to transduce a CHO cell panel, and luminescence in cell lysates was measured after 72 hours. The particles transduced each CHO cell line to a similar degree (FIG. 5B). CDV-F/H LVs displaying parental or amino acid substituted glycoproteins were then used to transduce the COH cell panel. The P493S/Y539A substitution eliminated tropism for canine nectin-4 (FIG. 5C, middle), while the V478L/L479D/T544S/T548D (LDSD) substitution eliminated tropism for both canine nectin-4 and canine slamf1 (FIG. 5C, right). CDV-F/H-LVs having the LDSD substitution was then retargeted to EpCAM by incorporating Ac1, an EpCAM-specific DARPin targeting ligand, at the C-terminus of the H polypeptide (FIG. 5D). Further tropism engineering studies targeted human SLAMF1. CDV-F/H-LVs displaying parental or substituted CDV-H polypeptide were used to transduce the CHO cell panel. Neither the D540G substitution nor the combination of S194I/V195R/V478L/L479D/T544S/T548D conferred tropism for human SLAMF1, but when the two sets of mutations were combined, tropism for human SLAMF1 was observed (FIG. 5E).

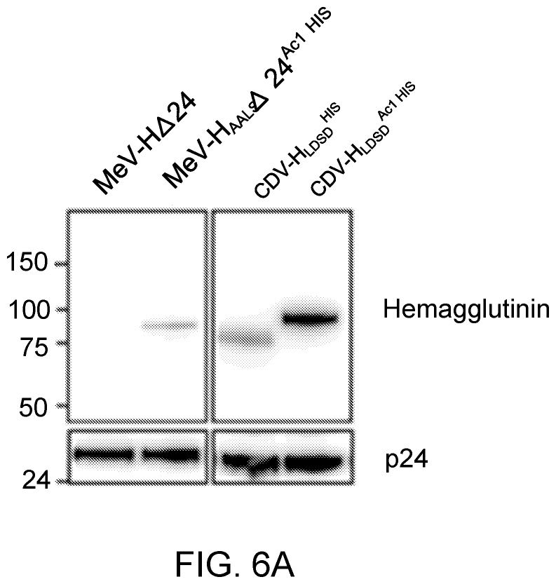

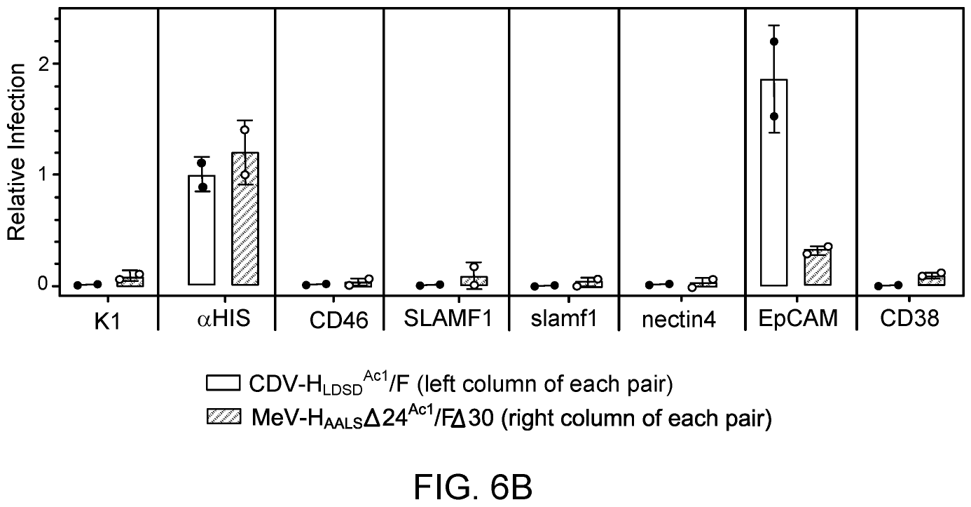

Western blot analysis of LVs particles for incorporation of MeV-HΔ24 in combination with MeV-FΔ30, MeV-HaalsΔ24HIS in combination with MeV-FΔ30, CDV-HLDSDHIS in combination with CDV-F, or CDV-HLDSDAc1 HIS in combination with CDV-F showed greater incorporation of the CDV-F/H polypeptides (FIG. 6A). In addition, receptor-dependent transduction of pseudotyped LVs on the parental CHO cell line (K1) or CHO cells stably expressing anti-HIS, CD46, human SLAMF, canine SLAMF1, canine nectin4, EpCAM, or CD38 demonstrated that targeting of the CDV-H_{LDSD}^{Ac1}/F LVs was significantly higher than that of the MeV counterpart (FIG. 6B).

For pseudotyping and retargeting of lentiviral entry using CDV-H and F polypeptides, an F/H combination less capable of triggering intercellular fusion demonstrated a considerably higher degree of retargeting than a highly fusogenic pairing (5804-22458/16) used to maximize intercellular fusion. This was only true, however, when the F/H combination was used with the modifications to the H and F polypeptide cytoplasmic tails described above, which are distinct from those described elsewhere for lentiviral targeting by measles F/H glycoproteins (Muñoz-Alía et al., PLoS Pathog 17(2):e1009283(2021). In the case of MeV polypeptides, truncations of more than 26 amino acids to the cytoplasmic tail of the H polypeptide reduced the efficiency of lentiviral vector targeting, possibly because these more extreme truncations impaired the fusion function of the F/H complex. In the case of CDV, a cytoplasmic tail truncation of 20 amino acids was associated with very low efficiency of lentiviral vector targeting, while a truncation of 30 amino acids was associated with highly efficient targeted lentiviral entry (FIG. 4E).

TABLE 4
Mutations for knocking out tropism

| Tropism | Amino acid change |
|---|---|
| Canine SLAM− | Y525A |
| | D526A |
| | Y529A |
| | Y525A/D526A |
| | Y525A/R529A |
| | D526A/R529A |
| | Y525A/D526A/R529A |
| | D526S/I527S/S528A/R529A |
| | D526S/I527S/S528A/R529A/Y547A/T548A |
| | V478L/L479P/T544S/T548P |
| | S194I/V195R/V478L/L479P/T544S/T548P |
| Human/Canine Nestin-4− | P454A |
| | L460A |
| | L479A |
| | I494A |
| | L510A |
| | Y520A |
| | Y537A |
| | Y539A |
| | P493S/Y539A |
| | D526S/I527S/S528A/R529A/Y547A/T548A |
| | V478L/L479P/T544S/T548P |
| | S194I/V195R/V478L/L479P/T544S/T548P |

TABLE 5
Types of mutations and their effects
on tropism, including human SLAM

| Amino acid change | Tropism |
|---|---|
| P454A | Canine SLAMF1+ |
| | Canine nectin4− |
| | Human nectin4− |
| L460A | Canine SLAMF1+ |
| | Canine nectin4− |
| | Human nectin4− |
| L479A | Canine SLAMF1+ |
| | Canine nectin4− |
| | Human nectin4− |
| I494A | Canine SLAMF1+ |
| | Canine nectin4− |
| | Human nectin4− |
| L510A | Canine SLAMF1+ |
| | Canine nectin4− |
| | Human nectin4− |
| Y520A | Canine SLAMF1+ |
| | Canine nectin4− |
| | Human nectin4− |
| Y537A | Canine SLAMF1+ |
| | Canine nectin4− |
| | Human nectin4− |
| Y539A | Canine SLAMF1+ |
| | Canine nectin4− |
| | Human nectin4− |
| P493S/Y539A | Canine SLAMF1+ |
| | Canine nectin4− |
| | Human nectin4− |
| Y525A | Canine SLAMF1− |
| | Human nectin4+ |
| | Canine nectin4+ |
| D526A | Canine SLAMF1− |
| | Canine nectin4+ |
| | Human nectin4+ |
| Y529A | Canine SLAMF1− |
| | Canine nectin4− |
| | Human nectin4− |
| D540G | Canine SLAMF1+ |
| | Canine nectin4− |
| | Human nectin4− |
| Y525A/D526A | Canine SLAMF1− |
| | Human nectin-4+ |
| | Canine nectin-4+ |
| Y525A/R529A | Canine SLAMF1− |
| | Human nectin-4+ |
| | Canine nectin-4+ |
| D526A/R529A | Canine SLAMF1− |
| | Human nectin-4+ |
| | Canine nectin-4+ |
| Y525A/D526A/R529A | Canine SLAMF1− |
| | Human nectin-4− |
| | Canine nectin-4− |
| D526S/I527S/S528A/R529A | Canine SLAMF1− |
| | Canine nectin4− |
| | Human nectin4− |
| D526S/I527S/S528A/R529A/Y547A/T548A | Canine SLAMF1− |
| | Canine nectin4− |
| | Human nectin4− |
| V478L/L479P/T544S/T548P | Canine SLAMF1− |
| | Canine nectin4− |
| | Human nectin4− |
| S194I/V195R/V478L/L479P/T544S/T548P | Canine SLAMF1− |
| | Canine nectin4− |
| | Human nectin4− |
| S194I/V195R/V478L/L479P/D540G/T544S/T548P | Canine SLAMF1+ |
| | Human SLAMF1+ |
| | Human nectin4− |
| | Canine nectin4− |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pseudotyped virus comprising a canine distemper virus (CDV) hemagglutinin (H) polypeptide and a CDV fusion (F) polypeptide, wherein said virus lacks nucleic acid encoding said H polypeptide and lacks nucleic acid encoding said F polypeptide.

2. The pseudotyped virus of claim 1, wherein said virus is a lentivirus (LV).

3. The pseudotyped virus of claim 1 or claim 2, wherein said CDV-H polypeptide comprises an amino acid substitution at one or more of positions 194, 195, 478, 479, 540, 544, and 548 according the amino acid numbering of SEQ ID NO:17.

4. The pseudotyped virus of claim 3, wherein said CDV-H polypeptide comprises V478L, L479D, T544S, and T548D substitutions according to the amino acid numbering of SEQ ID NO:17.

5. The pseudotyped virus of claim 3, wherein said CDV-H polypeptide comprises a D540G substitution according to the amino acid numbering of SEQ ID NO:17.

6. The pseudotyped virus of claim 3, wherein said CDV-H polypeptide comprises S194I, V195R, V478L, L479D, D540G, T544S, and T548D substitutions according to the amino acid numbering of SEQ ID NO:17.

7. The pseudotyped virus of any one of claims 1 to 6, wherein said CDV-H polypeptide comprises a truncated N-terminal cytoplasmic domain, as compared to the sequence set forth in SEQ ID NO:17.

8. The pseudotyped virus of claim 7, wherein said truncated N-terminal cytoplasmic domain comprises a deletion that is 27 to 32 amino acids in length.

9. The pseudotyped virus of claim 8, wherein said truncated N-terminal cytoplasmic domain has the sequence set forth in SEQ ID NO:23.

10. The pseudotyped virus of any one of claims 1 to 9, wherein said CDV-F polypeptide comprises the amino acid sequence set forth in SEQ ID NO:2.

11. The pseudotyped virus of any one of claims 1 to 9, wherein said CDV-F polypeptide comprises a signal peptide sequence that is less than 75 amino acid residues in length.

12. The pseudotyped virus of claim 11, wherein said signal peptide sequence comprises no more than 75 amino acid residues of SEQ ID NO:24.

13. The pseudotyped virus of claim 11 or claim 12, wherein said CDV-F polypeptide comprises SEQ ID NO:2 with the proviso that said CDV-F polypeptide lacks at least amino acid residues 1 to 60 or lacks at least amino acid residues 1 to 105 of SEQ ID NO:2.

14. The pseudotyped virus of any one of claims 1 to 13, wherein said virus is a lentivirus, and wherein nucleic acid within said lentivirus is disarmed.

15. The pseudotyped virus of any one of claims 1 to 14, wherein said virus is a lentivirus, and wherein said lentivirus comprises exogenous nucleic acid encoding one or more of an interferon (IFN) polypeptide, a sodium iodide symporter (NIS) polypeptide, a toxin polypeptide, or a chimeric antigen receptor (CAR) polypeptide.

16. The pseudotyped virus of any one of claims 1 to 15, wherein said CDV-H polypeptide comprises an amino acid sequence of a single chain antibody.

17. The pseudotyped virus of claim 16, wherein said single chain antibody is a single chain antibody that specifically binds to a CD19, CD20, CD38, CD46, CD117, EGFR, αFR, HER2/neu, PSMA, or EpCAM polypeptide.

18. A composition comprising a pseudotyped virus of any one of claims 1 to 17.

19. A CDV-H polypeptide comprising an amino acid substitution at one or more of positions 194, 195, 478, 479, 540, 544, and 548 according to the amino acid numbering of SEQ ID NO:17.

20. The CDV-H polypeptide of claim 19, wherein said polypeptide comprises V478L, L479D, T544S, and T548D substitutions according to the amino acid numbering of SEQ ID NO:17.

21. The CDV-H polypeptide of claim 19, wherein said polypeptide comprises a D540G substitution according to the amino acid numbering of SEQ ID NO:17.

22. The CDV-H polypeptide of claim 19, wherein said polypeptide comprises S194I, V195R, V478L, L479D, D540G, T544S, and T548D substitutions according to the amino acid numbering of SEQ ID NO:17.

23. The CDV-H polypeptide of any one of claims 19 to 22, wherein said polypeptide comprises a truncated N-terminal cytoplasmic domain, as compared to the sequence set forth in SEQ ID NO:17.

24. The CDV-H polypeptide of claim 23, wherein said truncated N-terminal cytoplasmic domain comprises a deletion that is 27 to 32 amino acids in length.

25. The CDV-H polypeptide of claim 24, wherein said truncated N-terminal cytoplasmic domain has the sequence set forth in SEQ ID NO:23.

26. The CDV-H polypeptide of any one of claims 19 to 25, wherein said polypeptide comprises an amino acid sequence of a single chain antibody.

27. The CDV-H polypeptide of claim 26, wherein said single chain antibody is a single chain antibody that specifically binds to a CD19, CD20, CD38, CD46, CD117, EGFR, αFR, HER2/neu, PSMA, or EpCAM polypeptide.

28. A nucleic acid molecule encoding a CDV-H polypeptide of any one of claims 19 to 28.

29. A composition comprising a nucleic acid molecule of claim 28.

30. The composition of claim 29, further comprising a nucleic acid molecule encoding a CDV-F polypeptide.

31. The composition of claim 30, wherein said CDV-F polypeptide comprises the amino acid sequence set forth in SEQ ID NO:2.

32. The composition of claim 30, wherein said CDV-F polypeptide comprises a signal peptide sequence that is less than 75 amino acid residues in length.

33. The composition of claim 32, wherein said signal peptide sequence comprises no more than 75 amino acid residues of SEQ ID NO:24.

34. The composition of claim 32 or claim 33, wherein said CDV-F polypeptide comprises SEQ ID NO:2 with the proviso that said CDV-F polypeptide lacks at least amino acid residues 1 to 60 or lacks at least amino acid residues 1 to 105 of SEQ ID NO:2.

35. A method for treating cancer, wherein said method comprises administering a composition of any one of claims 18 and 30 to 35 to a mammal comprising cancer cells, wherein the number of cancer cells within said mammal is reduced following said administration.

36. The method of claim 35, wherein said mammal is a human.

37. The method of claim 35 or claim 36, wherein said cancer is myeloma, melanoma, glioma, lymphoma, mesothelioma, lung cancer, brain cancer, stomach cancer, colon cancer, rectum cancer, kidney cancer, prostate cancer, ovary cancer, breast cancer, pancreas cancer, liver cancer, or head and neck cancer.

38. A method for inducing tumor regression in a mammal, wherein said method comprises administering a composition of any one of claims 18 and 29 to 34 to a mammal comprising a tumor, wherein the size of said tumor is reduced following said administration.

39. The method of claim 38, wherein said mammal is a human.

40. The method of claim 38 or claim 39, wherein said cancer is myeloma, melanoma, glioma, lymphoma, mesothelioma, lung cancer, brain cancer, stomach cancer, colon cancer, rectum cancer, kidney cancer, prostate cancer, ovary cancer, breast cancer, pancreas cancer, liver cancer, or head and neck cancer.
